(19) United States
(12) Patent Application Publication
(10) Pub. No.: US 2010/0155242 A1
(43) Pub. Date: Jun. 24, 2010

(54) Method of Analyzing a Sample by Capillary Electrophoresis

(75) Inventors: Yusuke Nakayama, Kyoto (JP); Koji Sugiyama, Kyoto (JP); Yoshihide Tanaka, Osaka (JP); Shinichi Wakida, Osaka (JP); Satoshi Yonehara, Kyoto (JP)

Correspondence Address: MORGAN LEWIS & BOCKIUS LLP, 1111 PENNSYLVANIA AVENUE NW, WASHINGTON, DC, 20004, US

(73) Assignees: ARKRAY, Inc., Kyoto (JP); NATIONAL INSTITUTE OF ADVANCED INDUSTRIAL SCIENCE AND TECHNOLOGY, Tokyo (JP)

(21) Appl. No.: 12/635,472

(22) Filed: Dec. 10, 2009

Related U.S. Application Data
(63) Continuation-in-part of application No. 12/367,260, filed on Feb. 6, 2009.
Application No. 12/367,260 is a continuation-in-part of application No. 12/376,739, filed on Mar. 20, 2009.
Application No. 12/376,739 is a continuation-in-part of application No. 12/376,744, filed on Mar. 20, 2009.

(30) Foreign Application Priority Data
Sep. 4, 2006 (JP) JP 2006-239640
Sep. 4, 2006 (JP) JP 2006-239641
Feb. 8, 2008 (JP) JP 2008-029751

(51) Int. Cl. C07K 1/26; G01N 27/00

(52) U.S. Cl. 204/451; 204/601

(57) ABSTRACT

The present invention is directed to the described capillary electrophoresis apparatus and methods of using such apparatus for separating and analyzing components of a sample.

### RELATED APPLICATIONS

This application is a continuation-in-part of each of U.S. application Ser. No. 12/367,260, filed Feb. 6, 2009, which claims the benefit of Japanese Patent Application JP 2008-029751, filed Feb. 8, 2008; U.S. application Ser. No. 12/376,739, filed Mar. 20, 2009, which is a National Stage of International Application PCT/JP2007/066751, filed on Aug. 29, 2007, which claims the benefit of Japanese Patent Application JP 2006-239640, filed Sep. 4, 2006; and U.S. application Ser. No. 12/376,744, filed Mar. 20, 2009, which is a National Stage of International Application PCT/JP2007/066752, filed on Aug. 29, 2007, which claims the benefit of Japanese Application 2006-239642, filed Sep. 4, 2006. All patent applications cited herein are incorporated by reference in their entireties.

### FIELD OF THE INVENTION

The present invention relates to the described capillary electrophoresis apparatus and methods of using such apparatus for separating components of a sample.

### BACKGROUND OF THE INVENTION

Diabetes is a metabolic disorder characterized by abnormally high blood sugar levels or hyperglycemia. Diabetes is diagnosed and/or monitored by measuring blood glucose levels in a patient. In recent years, a test known as the A1C test was developed to measure the average amount of blood glucose in a patient for the past few months prior to the test. The A1C test measures glycated hemoglobin.

Glycated hemoglobin is formed when hemoglobin (Hb) reacts with glucose in the blood. There are different types of glycated Hb that occur in the bloodstream. One type of glycated hemoglobin, hemoglobin A1c (HbA1c), is used as an important indicator in the diagnosis and treatment of diabetes. HbA1c has a chemical structure in which an N-terminal valine of the β-chain of hemoglobin A (HbA0) is glycated. Stable and unstable forms of HbA1c exist in the bloodstream. Whether HbA1c is in a stable or unstable form depends on the stage of the glycation reaction. HbA0 becomes unstable HbA1c when a N-terminal valine of the β-chain of HbA0 is reacted with glucose, and the glucose reacts with Hb to form an aldimine (e.g., Schiff base). Unstable HbA1c becomes stable HbA1c when the aldimine is changed to a ketoamine group by an Amadori rearrangement. The level of stable HbA1c in blood is an indicator of the glucose levels that have been present in a patient's blood for a few months prior to testing, and its measurement for the treatment and diagnosis of diabetes is endorsed by The Japan Diabetes Society.

Examples of methods that can be used to determine glycated hemoglobin levels in blood include immunoassays, enzymatic methods, affinity chromatography methods, HPLC (high pressure liquid chromatography or high performance liquid chromatography) methods, and capillary electrophoresis (CE) methods, among others. Because the immunoassay methods and the enzymatic methods can be performed using an autoanalyzer, they have the advantage of being able to readily handle a large quantity of specimens. However, the immunoassay methods and the enzymatic methods lack sufficient measurement accuracy to be relied on by diabetes patients as a blood glucose control indicator (preventive marker for onset of complications). Further, in principle, affinity chromatography methods have only low specificity for the glycated valine of the β-chain N-terminal in HbA1c, and thus, glycated lysine residues in Hb molecules can interfere with the making of accurate measurements. Therefore, the measurement accuracy of HbA1c by affinity chromatography methods is low.

HPLC methods are widely used to determine glycated hemoglobin levels for diabetes patients (see, for example, JP 3429709 B). However, HPLC methods require specialized instruments that are large and expensive. In order for HPLC methods to be practical for the analysis of groups of samples (as in a clinical laboratory), the hemoglobin analyzer would have to be downsized. It would be difficult to reduce the size and cost of such instruments. In contrast, capillary electrophoresis instruments require less space. In capillary electrophoresis, ions that have gathered on the inner wall of a capillary channel move in response to an application of voltage, creating an electroosmotic flow which results in separation of components of a sample, and thus electrophoresis is performed. The capillary electrophoresis channel may be made of fused silica. Due to the chemical nature of the fused silica, some analytes are adsorbed to it and adhere to the inner wall of the capillary. This phenomenon can cause electroosmotic flow to be impeded and may contribute to imprecise results.

To address this problem, various methods of coating the inner wall of the capillary channel have been proposed (see, for example, JP 2005-291926 A, JP 4(1992)-320957 A, JP 5(1993)-503989 A and JP 8(1996)-504037 A). For example, a protein can be used to coat the inner wall of the capillary channel that is used to electrophorese hemoglobin, and the protein coating can in turn be coated with a polysaccharide (JP 9(1997)-105739 A). A drawback of this protein-coating method is that the protein has to be re-applied to the inner wall of the capillary channel each time the capillary channel is used. Another approach to overcoming the problems associated with the adsorption of analytes by fused silica involves using a electrophoresis buffer that is zwitterionic and contains a flow inhibitor (i.e., an aliphatic diamine), instead of coating the inner wall (see JP 2006-145537 A). However, while this approach permits separation of modified hemoglobins, it does not permit separation of glycated hemoglobins. With respect to the capillary electrophoresis method, CE instruments can be downsized by reducing the length of the capillary channel and by microchipping a part of a capillary electrophoresis apparatus.

### SUMMARY OF THE INVENTION

An aspect of the invention is a capillary channel as described herein, wherein the inner wall of the capillary channel is coated with a coating comprising a cationic layer or an anionic layer.

Another aspect of the invention is a capillary channel as described herein, wherein the inner wall of the capillary channel is coated with a coating comprising an A layer and a B layer, wherein the A layer comprises a cationic layer or a nonpolar layer, and the B layer comprises an anionic layer, and wherein the B layer covers the A layer, the A layer being closer to the inner wall of the capillary channel than the B layer.

Another aspect of the invention is a capillary electrophoresis apparatus comprising a capillary channel as described herein.

Another aspect of the invention is a method of analyzing a sample comprising applying a sample to a capillary electrophoresis apparatus comprising a coated capillary channel as described herein; and performing electrophoretic separation of the sample, wherein the capillary channel contains an electrophoresis buffer solution.

Another aspect of the invention is a method of analyzing a sample comprising applying a sample to a capillary electrophoresis apparatus comprising an uncoated capillary channel; and performing electrophoretic separation of the sample, wherein the capillary channel contains an electrophoresis buffer solution and wherein an anionic group-containing compound is present in the buffer solution, the sample or combinations thereof during at least a portion of the electrophoretic separation.

Another aspect of the invention is a method of diagnosing diabetes in a subject comprising obtaining a sample of blood from a subject; applying the sample to a capillary electrophoresis apparatus comprising a coated capillary channel as described herein; and performing electrophoretic separation of the sample for determining the amount of glycated hemoglobin in the sample, thereby determining whether the subject has diabetes, wherein the capillary channel contains an electrophoresis buffer solution.

Another aspect of the invention is a method of diagnosing diabetes in a subject comprising obtaining a sample of blood from a subject; applying the sample to a capillary electrophoresis apparatus comprising an uncoated capillary channel; and performing electrophoretic separation of the sample for determining the amount of glycated hemoglobin in the sample, thereby determining whether the subject has diabetes, wherein the capillary channel contains an electrophoresis buffer solution and wherein an anionic group-containing compound is present in the buffer solution, the sample or combinations thereof during at least a portion of the electrophoretic separation.

Another aspect of the invention is a method of monitoring diabetes in a subject comprising obtaining a sample of blood from a subject; applying the sample to a capillary electrophoresis apparatus comprising a coated capillary channel as described herein; and performing electrophoretic separation of the sample for determining the amount of glycated hemoglobin in the sample, thereby determining whether the subject has diabetes, wherein the capillary channel contains an electrophoresis buffer solution.

Another aspect of the invention is a method of monitoring diabetes in a subject comprising obtaining a sample of blood from a subject; applying the sample to a capillary electrophoresis apparatus comprising an uncoated capillary channel; and performing electrophoretic separation of the sample for determining the amount of glycated hemoglobin in the sample, thereby determining whether the subject has diabetes, wherein the capillary channel contains an electrophoresis buffer solution and wherein an anionic group-containing compound is present in the buffer solution, the sample or combinations thereof during at least a portion of the electrophoretic separation.

Another aspect of the invention is a kit for diagnosing or monitoring diabetes in a subject comprising a container for obtaining blood from a subject and at least one capillary electrophoresis buffer solution, wherein the buffer solution comprises an anionic group-containing compound.

In an exemplary embodiment, the cationic layer comprises amino groups or salts thereof, ammonium groups or mixtures thereof.

In an exemplary embodiment, the anionic layer comprises sulfate groups, carboxylate groups, sulfonate groups, phosphate groups or mixtures thereof.

In an exemplary embodiment, the anionic layer comprises a chaotropic anion.

In an exemplary embodiment, the inner diameter of the capillary channel is about 10 μm and about 200 μm.

In an exemplary embodiment, the cationic layer comprises a polydiallydimethylammonium group.

In an exemplary embodiment, the anionic layer comprises an anionic group-containing polysaccharide.

In an exemplary embodiment, the polysaccharide of the anionic group-containing polysaccharide is a sulfated polysaccharide, a carboxylated polysaccharide, a sulfonated polysaccharide, a phosphorylated polysaccharide or mixtures thereof.

In an exemplary embodiment, the nonpolar layer comprises polysiloxanes, polysilazanes or mixtures thereof.

In an exemplary embodiment, the capillary electrophoresis apparatus further comprising a substrate and a plurality of liquid tanks, wherein the liquid tanks are allowed to communicate with each other through the capillary channel.

In an exemplary embodiment, an anionic group-containing compound is present in the buffer solution, the sample or combinations thereof during at least a portion of the electrophoretic separation.

In an exemplary embodiment, the anionic group-containing compound is a chaotropic anion, a sulfated polysaccharide, a carboxylated polysaccharide, a sulfonated polysaccharide, a phosphorylated polysaccharide or mixtures thereof.

In an exemplary embodiment, the chaotropic anion is perchlorate, thiocyanate, trichloroacetate, trifluoroacetate, nitrate, dichloroacetate, halogenide or mixtures thereof.

In an exemplary embodiment, a chaotropic anion is present in the buffer solution, the sample or combinations thereof during at least a portion of the electrophoretic separation.

In an exemplary embodiment, the sample comprises hemoglobin.

### BRIEF DESCRIPTION OF THE DRAWINGS

The following figures are simply illustrative of exemplary embodiments of the invention and are not intended to otherwise restrict the scope of the disclosure.

In each electropherogram of FIGS. 1 to 20, the vertical (y-) axis corresponds to absorbance measured at 415 nm and the horizontal (x-) axis corresponds to time in minutes.

FIG. 1 is an electropherogram showing the analysis result of hemoglobin in Example 1 as described herein. The peaks indicated by arrows are, from left to right, unstable HbA1c, stable HbA1c, and HbA0.

FIG. 2 is an electropherogram showing the analysis result of hemoglobin in Example 2 as described herein. The peaks indicated by arrows are, from left to right, unstable HbA1c, stable HbA1c, and HbA0.

FIG. 3 is an electropherogram showing the analysis result of hemoglobin in Example 3 as described herein. The peaks indicated by arrows are, from left to right, unstable HbA1c, stable HbA1c, and HbA0.

FIG. 4 is an electropherogram showing the analysis result of hemoglobin in Example 4 as described herein. The peaks indicated by arrows are, from left to right, unstable HbA1c, stable HbA1c, and HbA0.

FIG. 5 is an electropherogram showing the analysis result of hemoglobin in Example 5 as described herein. The peaks indicated by arrows are, from left to right, unstable HbA1c, stable HbA1c, and HbA0.

FIG. 6 is an electropherogram showing the analysis result of hemoglobin in Example 6 as described herein. The peaks indicated by arrows are, from left to right, unstable HbA1c, stable HbA1c, and HbA0.

FIG. 7 is an electropherogram showing the result of analysis of hemoglobin in Example 7 as described herein. The peaks indicated by arrows are, from left to right, HbA1c and HbA0.

FIG. 8 is an electropherogram showing the result of analysis of hemoglobin in Example 8 as described herein. The peaks indicated by arrows are, from left to right, HbA1c and HbA0.

FIG. 9 is an electropherogram showing the result of analysis of hemoglobin in Example 9 as described herein. The peaks indicated by arrows are, from left to right, HbA1c and HbA0.

FIG. 10 is an electropherogram showing the result of analysis of hemoglobin in Example 10 as described herein. The peaks indicated by arrows are, from left to right, HbA1c and HbA0.

FIG. 11 is an electropherogram showing the result of analysis of hemoglobin in Example 11 as described herein. The peaks indicated by arrows are, from left to right, HbA1c and HbA0.

FIG. 12 is an electropherogram showing the result of analysis of hemoglobin in Example 12 as described herein. The peaks indicated by arrows are, from left to right, HbA1c and HbA0.

FIG. 13 is an electropherogram showing the result of analysis of hemoglobin in Example 13 as described herein. The peaks indicated by arrows are, from left to right, unstable HbA1c, stable HbA1c, and HbA0. The peaks indicated by arrows are, from left to right, HbA1c and HbA0.

FIG. 14 is an electropherogram showing the analysis result of hemoglobin in Example 14 which is a comparative example as described herein. The peaks indicated by arrows are, from left to right, unstable HbA1c, stable HbA1c, and HbA0.

FIG. 15 is an electropherogram showing the analysis result of hemoglobin in Example 15 which is a comparative example as described herein. The peaks indicated by arrows are, from left to right, unstable HbA1c, stable HbA1c, and HbA0.

FIG. 16 is an electropherogram showing the analysis result of hemoglobin in Example 16 which is a comparative example as described herein. The peak indicated by an arrow is HbA0.

FIG. 17 is an electropherogram showing the analysis result of hemoglobin in Example 17 as described herein. The peaks indicated by arrows are, from left to right, carbamoylated Hb and stable HbA1c.

FIG. 18 is an electropherogram showing the analysis result of hemoglobin in Example 18 which is a comparative example as described herein. The peaks indicated by arrows are, from left to right, carbamoylated Hb and stable HbA1c.

FIG. 19 is an electropherogram showing the analysis result of hemoglobin in Example 19 as described herein. The peaks indicated by arrows are, from left to right, acetylated Hb and stable HbA1c.

FIG. 20 is an electropherogram showing the analysis result of hemoglobin in Example 20 which is a comparative example as described herein. The peaks indicated by arrows are, from left to right, acetylated Hb and stable HbA1c.

FIG. 21 shows diagrams illustrating the configuration of an exemplary embodiment of the capillary electrophoresis apparatus of the invention. FIG. 21(A) is a plan view of the capillary electrophoresis apparatus of this example, FIG. 21(B) is a sectional view taken on line I-I shown in FIG. 21(A), and FIG. 21(C) is a sectional view taken on line II-II shown in FIG. 21(A).

FIG. 22 shows diagrams illustrating the configuration of another exemplary embodiment of the capillary electrophoresis apparatus of the invention.

FIG. 23 shows diagrams illustrating the configuration of another exemplary embodiment of the capillary electrophoresis apparatus of the invention.

FIG. 24 shows diagrams illustrating the configuration of yet another exemplary embodiment of the capillary electrophoresis apparatus of the invention.

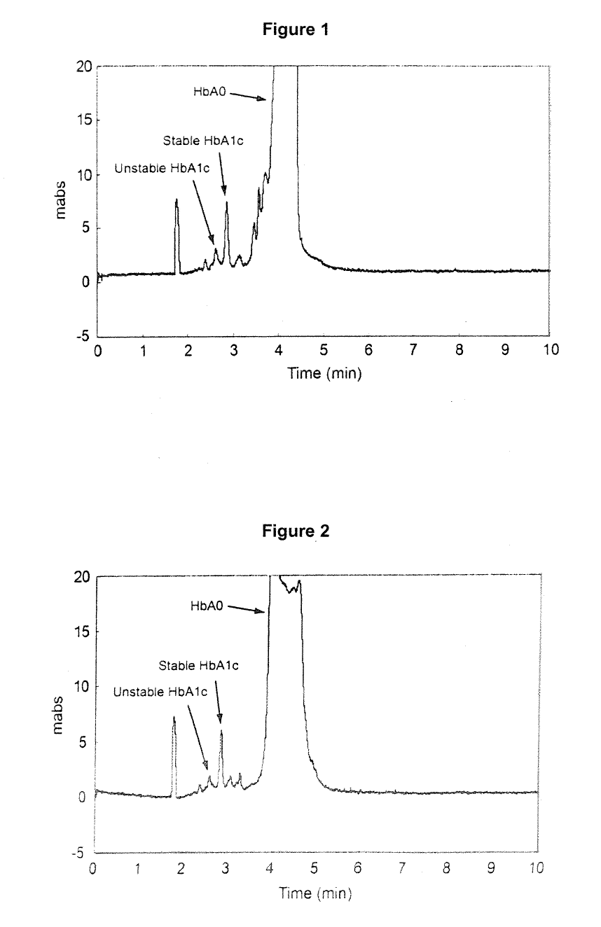

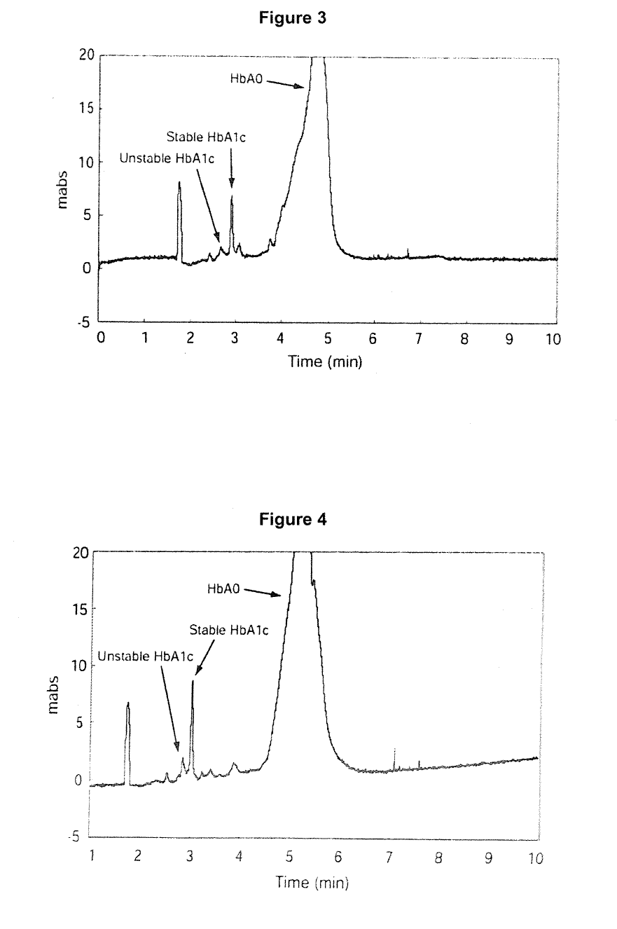

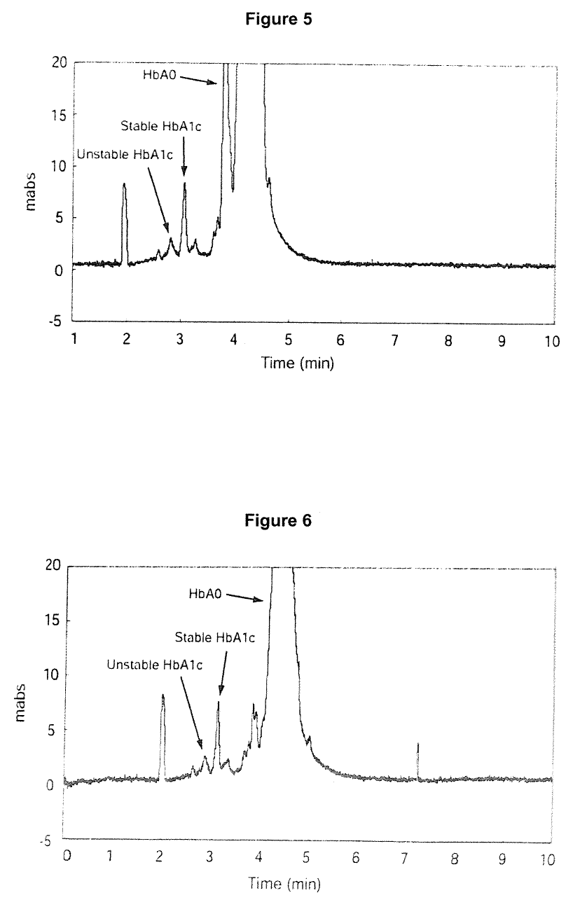

### DETAILED DESCRIPTION

An aspect of the invention is a CE apparatus, comprising a capillary channel as described herein. In an exemplary embodiment, the CE apparatus is a reduced size microchip electrophoresis apparatus as described herein. The CE apparatus of the invention is described using the following examples. However, the examples merely represent particular embodiments of the CE apparatus and are not intended to further limit the scope of the invention as disclosed herein. Accordingly, a CE apparatus of the invention may be structured differently from those specifically described in the examples and may be produced by processes other than those specifically recited below.

The CE apparatus of the invention may include a substrate, a plurality of liquid tanks and a capillary channel, wherein the plurality of liquid tanks may be formed in the substrate and may be allowed to communicate with one another through the capillary channel. In an exemplary embodiment of the invention, the substrate has a maximum length in the range of about 10 to about 100 mm, such as about 30 to about 70 mm; a maximum width in the range of about 10 to about 60 mm; and a maximum thickness in the range of about 0.3 to about 5 mm. In an exemplary embodiment of the invention, the maximum length of the substrate is the length of the portion that is longest in the longitudinal direction of the substrate; the maximum width of the substrate is the length of the portion that is longest in the direction (width direction) perpendicular to the longitudinal direction of the substrate; and the maximum thickness of the substrate is the length of the portion that is longest in the direction (thickness direction) perpendicular to both the longitudinal direction and the width direction of the substrate.

FIG. 21 shows an example of a CE apparatus according to the invention. FIG. 21(A) is a plan view of the CE apparatus. FIG. 21(B) is a sectional view taken on line I-I shown in FIG. 21(A), and FIG. 21(C) is a sectional view taken on line II-II shown in FIG. 21(A). The CE apparatus of this example is a microchip electrophoresis apparatus with a reduced size (formed into a microchip). As depicted, this microchip electrophoresis apparatus includes a substrate 1, a plurality of four liquid tanks 2a to 2d, and four capillary channels 3x1, 3x2, 3y1, and 3y2. The four liquid tanks 2a to 2d include a first introduction tank 2a, a first recovery tank 2b, a second introduction tank 2c, and a second recovery tank 2d. In the four capillary channels, one end of each of the channels meets at the central portion c to be joined together in a cross shape. Accordingly, the four capillary channels communicate with one another by their inner parts. The substrate 1 is provided with a cavity for inserting the four capillary channels thereinto (not shown in the figures). The capillary channel 3x1 is inserted into the substrate 1 so that the other end thereof is located at the bottom surface of the first introduction tank 2a. The capillary channel 3x is inserted into the substrate 1 so that the other end thereof is located at the bottom surface of the first recovery tank 2b. The capillary channels 3x1 and 3x2 form a capillary channel 3x for sample analysis. The capillary channel 3y1 is inserted into the substrate 1 so that the other end thereof is located at the bottom surface of the second introduction tank 2c. The capillary channel 3y2 is inserted into the substrate 1 so that the other end thereof is located at the bottom surface of the second recovery tank 2d. The capillary channels 3y1 and 3y2 form a capillary channel 3y for sample introduction. The plurality of liquid tanks 2a to 2d is each formed as a concave part in the substrate 1. The substrate 1 has a rectangular parallelepiped opening (window) 9 on the first recovery tank 2b side with respect to the capillary channel 3y for sample introduction. While the microchip electrophoresis apparatus of this example is rectangular parallelepiped, the apparatus as described herein is not so limited. The microchip electrophoresis apparatus of the present invention may have any shape as long as it does not present problems in the electrophoresis measurement.

The inner diameters of the four capillary channels in FIG. 21 are the same as those of the capillary channels generally described herein. For example, the capillary channel 3x for sample analysis and the capillary channel 3y for sample introduction each may have in an exemplary embodiment a maximum length in the range of about 0.5 to about 15 cm. The respective lengths of the four capillary channels are determined according to the maximum lengths of the capillary channel 3x for sample analysis and the capillary channel 3y for sample introduction. In the microchip electrophoresis apparatus of FIG. 21, the capillary channel 3x for sample analysis is different in maximum length from the capillary channel 3y for sample introduction. However, the apparatus as generally described herein is not so limited. For example, in the microchip electrophoresis apparatus of the invention, the maximum length of the capillary channel 3x for sample analysis may be identical to that of the capillary channel 3y for sample introduction. Similarly with respect to other features of the apparatus of this example, the configuration of the microchip electrophoresis apparatus of the invention as generally described is not so limited.

The volumes of the plurality of liquid tanks 2a to 2d are not particularly limited. For example, each of them may have a volume of about 1 to about 1000 mm³, such as about 50 to about 100 mm³. In FIG. 21, the shapes of the plurality of liquid tanks 2a to 2d are cylindrical. However, the invention as described is not so limited. In the microchip electrophoresis apparatus of the invention, the shapes of the plurality of liquid tanks are not particularly limited as long as they do not present problems in the introduction and recovery of the sample described herein. For example, each of the tanks may have an arbitrary shape, such as a quadrangular prism shape, a quadrangular pyramidal shape, a conical shape, or a shape formed by combining these features. Furthermore, the volumes and shapes of the plurality of liquid tanks may be identical to or different from one another.

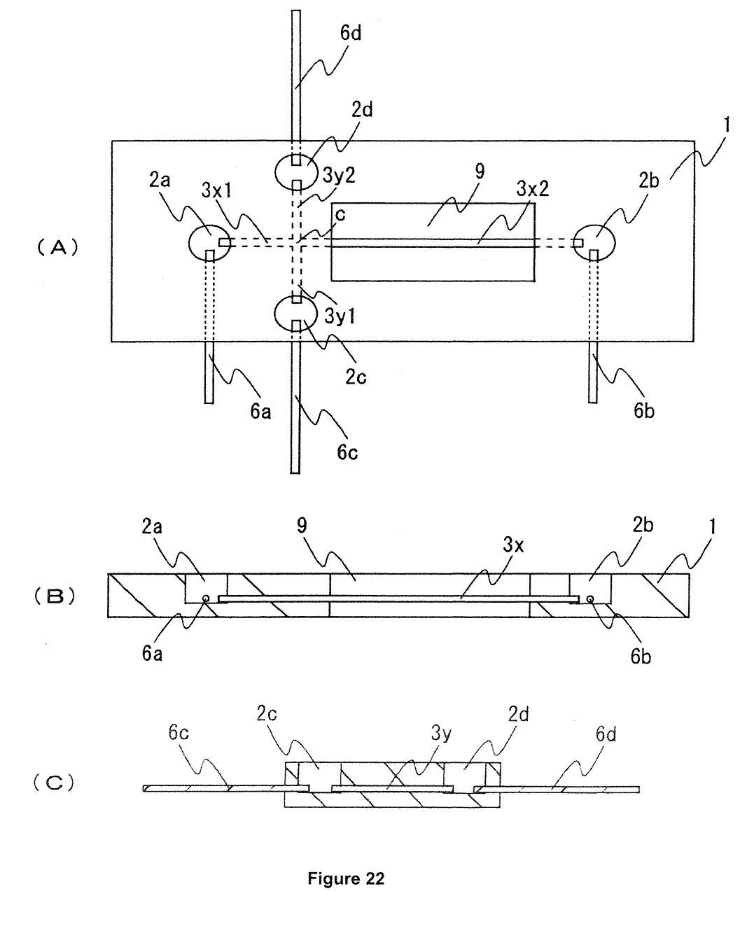

In the microchip electrophoresis apparatus of the invention, a plurality of electrodes is an optional component. FIG. 22 depicts a microchip electrophoresis apparatus that includes a plurality of electrodes (i.e., four electrodes 6a to 6d). In FIG. 22, the identical parts to those shown in FIG. 21 are indicated with identical numerals and symbols. The four electrodes 6a to 6d are buried in the substrate 1 in such a manner that one end of each of the electrodes is located inside the plurality of liquid tanks 2a to 2d, respectively. The four electrodes 6a to 6d can be disposed easily when, for example, holes for introducing the four electrodes 6a to 6d are formed in the side faces of the substrate 1 in producing the substrate 1. As an example, the plurality of electrodes may be inserted into the plurality of liquid tanks when the microchip electrophoresis apparatus is used. The electrodes 6a to 6d may be any electrodes, as long as they can be used for the electrophoresis method described herein. Exemplary electrodes include, but are not limited to, those made of stainless steel (SUS), platinum (Pt) or gold (Au).

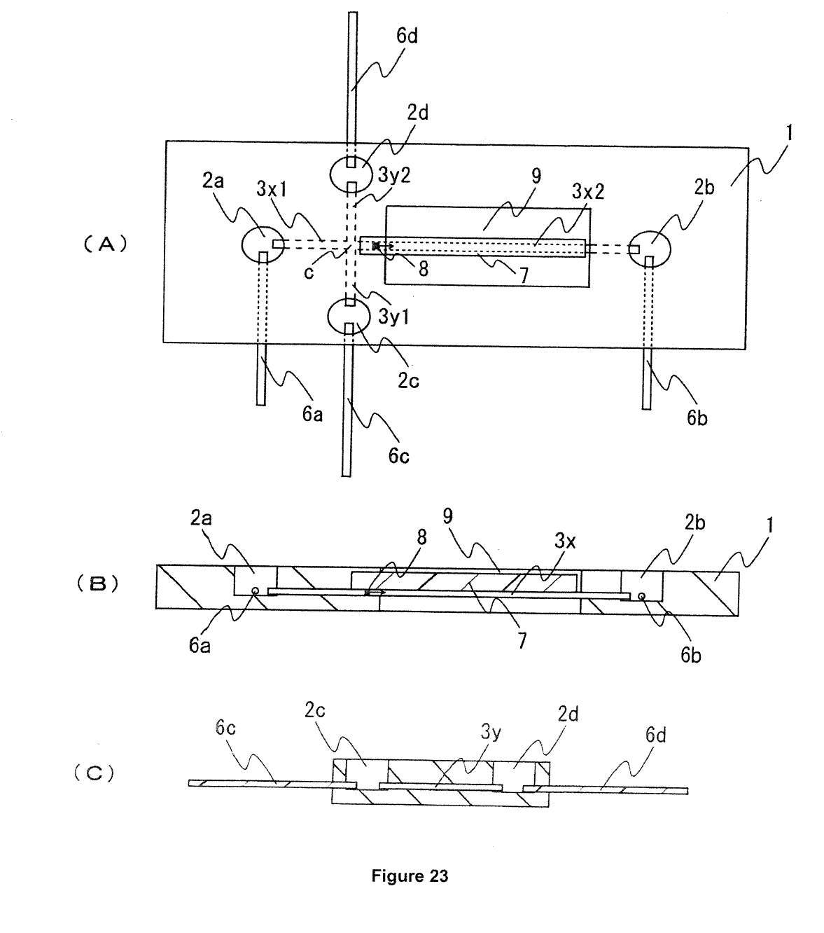

The microchip electrophoresis apparatus further may include an analysis unit. FIG. 23 depicts a microchip electrophoresis apparatus according to the invention that includes an analysis unit. In FIG. 23, identical parts to those shown in FIGS. 21 and 22 are indicated with identical numerals and symbols. As shown in FIG. 23, the microchip electrophoresis apparatus includes an analysis unit 7. In the microchip electrophoresis apparatus of this example, the analysis unit 7 is a detector (line detector). The line detector is disposed directly on the capillary channel 3x in such a manner that it is located on the first recovery tank 2b side with respect to the intersection part between the capillary channel 3x for sample analysis and the capillary channel 3y for sample introduction. In this microchip electrophoresis apparatus, the substrate 1 is provided with a cavity into which the analysis unit (line detector) 7 is to be inserted, in addition to the cavity into which the four capillary channels are to be inserted (not shown in the figures). The line detector has a light source and a detection unit built-in. The line detector emits light from the light source towards the sample to detect light reflected from the sample in the detection unit, and thereby measures absorbance. The analysis unit 7 is not limited to the line detector and may be any analysis unit as long as, for example, it can analyze a sample containing hemoglobin. For example, the analysis unit 7 may be configured with a light source disposed under the microchip electrophoresis apparatus and a detection unit disposed in a place corresponding to the place where the line detector is disposed. In this particular embodiment, light is emitted from the light source toward the sample, the transmitted light from the sample is detected in the detection unit, and thus absorbance is measured.

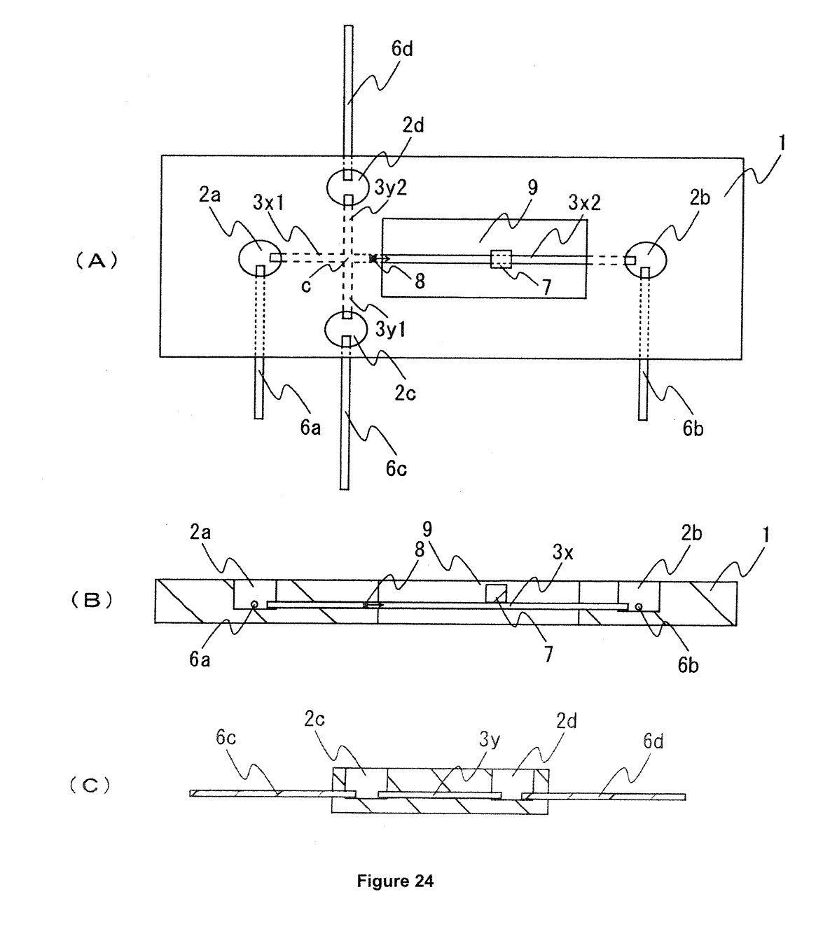

FIG. 24 shows still another example of the microchip electrophoresis apparatus according to the invention. In FIG. 24, identical parts to those shown in FIG. 23 are indicated with identical numerals and symbols. As depicted in FIG. 24, the microchip electrophoresis apparatus of this example has the same configuration as that of the microchip electrophoresis apparatus shown in FIG. 23 except that the analysis unit 7 is different. As in the example of FIG. 23, the analysis unit 7 may measure the absorbance at one point.

In the particular embodiment where the sample contains hemoglobin, the microchip electrophoresis apparatus may further include a pretreatment tank for hemolyzing the sample containing hemoglobin and diluting it. The treatment for hemolyzing the hemoglobin-containing sample is not particularly limited. For example, it may be a treatment for hemolyzing the hemoglobin-containing sample with a hemolytic agent. The hemolytic agent destroys, for example, a blood cell membrane of a blood cell component in the hemoglobin-containing sample. Examples of the hemolytic agent include, but are not limited to, the described running buffer, saponin, and Triton X-100™ manufactured by Nacalai Tesque, Inc. In a particular embodiment, the hemolytic agent is the running buffer. In an exemplary embodiment, the pretreatment tank communicates with, for example, the introduction tanks. The pretreatment tank may be formed in a suitable place such as near the liquid tank with which it communicates, for example, the second introduction tank 2c. When the pretreatment tank is provided, the hemoglobin-containing sample is introduced into the pretreatment tank. The hemoglobin-containing sample thus pretreated is introduced into a liquid tank that communicates with the pretreatment tank, for example, the second introduction tank 2c through the channel connecting the pretreatment tank and the second introduction tank 2c. The pretreatment tank may have a configuration in which two tanks, a tank for hemolyzing the hemoglobin-containing sample and a tank for diluting the hemoglobin-containing sample, are in communication with each other.

The methods of forming the four liquid tanks 2a to 2d and the opening (window) 9 in the substrate 1 (as shown in FIGS. 21-24) are not particularly limited. The substrate 1 may be formed of, for example, a glass or polymer material. Examples of the glass material include, but are not limited to, synthetic silica glass, and borosilicate glass. Examples of the polymer material include, but are not limited to, polymethylmethacrylate (PMMA), cycloolefin polymer (COP), polycarbonate (PC), polydimethylsiloxane (PDMS), polystyrene (PS), and polylactic acid. When the material used for the substrate 1 is a glass, the formation method can be, for instance, ultrasonic machining When the material used for the substrate 1 is a polymer material, the formation method can be, for example, a cutting method or a molding method such as injection molding, cast molding, or press molding that employs a mold. The four liquid tanks 2a to 2d and the opening (window) 9 each may be formed independently, or all of them may be formed simultaneously. When the four liquid tanks 2a to 2d and the opening (window) 9 each are formed independently, they may be formed in any order. In an exemplary embodiment, all four liquid tanks 2a to 2d and the opening (window) 9 are formed simultaneously by, for example, a method that employs a mold, since the number of the steps is smaller in this case. Next, the four capillary channels are inserted into the substrate 1. Thus, a microchip electrophoresis apparatus of this embodiment can be obtained.

The capillary channel used in the invention is not particularly limited, and may be a capillary tube or a capillary channel, such as one formed from the substrate of a microchip. The capillary channel may be prepared by the person performing the analysis, or alternatively, a commercially available device having a capillary channel may be used.

The material comprising the capillary channel is not particularly limited. Examples include, but are not limited to, glass, molten or fused silica, and polymeric materials (e.g., a plastic). When the capillary channel is made of glass or molten silica, the inner wall of the channel typically has a negative electric charge. The inner wall of a capillary channel made of plastic has a positive or negative electric charge depending on the presence or absence of polar groups and the type of the polar group contained in the plastic. Alternatively, the inner wall of the capillary channel is uncharged. This may be due to the presence of nonpolar groups. Even in the case of a plastic having no polar group, introduction of a polar group may result in the creation of electric charges. In an exemplary embodiment, a commercial product is used as the capillary channel. Examples of the capillary channel include, but are not limited to, those formed of synthetic silica glass, borosilicate glass, polymethylmethacrylate (PMMA), polycarbonate (PC), polystyrene (PS), polyethylene (PE), polytetrafluoroethylene (PTFE), polyetheretherketone (PEEK), cycloolefin polymer (COP), polydimethylsiloxane (PDMS), polylactic acid or mixtures thereof.

The inner diameter of the capillary channel is not particularly limited. In exemplary embodiments of the invention, the capillary channel used in electrophoresis may have an inner diameter between about 10 μm and about 200 μm, such as between about 15 μm and about 150 μm, such as between about 25 μm and about 100 μm. The length of a capillary channel is also not particularly limited. In an exemplary embodiment, the effective length of the capillary channel is the distance from the point where the sample begins electrophoresis in the capillary channel to the point along the capillary channel where the sample may be detected. In exemplary embodiments of the invention, the capillary channel may have a length of less than about 15 cm, less than about 10 cm, less than about 5 cm, between about 2 cm and about 3 cm, between about 10 mm and about 1000 mm, or between about 15 mm and about 300 mm.

In various exemplary embodiments of the invention, the capillary channel may be uncoated (e.g., a capillary channel without a coating attached to its inner wall) or coated with a coating agent comprising a cationic group, an anionic group or a combination of a cationic group and an anionic group.

In an exemplary embodiment of the invention, a compound comprising a cationic group and a reactive group may be used to coat the inner surface of a capillary channel. A capillary channel made of, for example, glass or fused silica may be coated with a compound containing a cationic group and at least one of silicon (e.g., a silylation agent), titanium, and zirconium. In an exemplary embodiment, the coating agent for a capillary channel may be a silylation agent having at least one cationic group. The cationic group may be, but is not limited to, an ammonium group, a primary amino group, a secondary amino group, a tertiary amino group or salts thereof. Examples of silylation agents having a cationic group that may be used as a coating agent include, but are not limited to, N-(2-diaminoethyl)-3-propyltrimethoxysilane, aminophenoxydimethylvinylsilane, 3-aminopropyldiisopropylethoxysilane, 3-aminopropylmethylbis(trimethylsiloxy)silane, 3-aminopropylpentamethyldisiloxane, 3-aminopropylsilanetriol, bis(p-aminophenoxy)dimethylsilane, 1,3-bis(3-aminopropyl)tetramethyldisiloxane, bis(dimethylamino)dimethylsilane, bis(dimethylamino)vinylmethylsilane, bis(2-hydroxyethyl)-3-aminopropyltriethoxysilane, 3-cyanopropyl(diisopropyl)dimethylaminosilane, (aminoethylaminomethyl)phenethyltrimethoxysilane, N-methylaminopropyltriethoxysilane, tetrakis(diethylamino)silane, tris(dimethylamino)chlorosilane, tris(dimethylamino)silane, and mixtures thereof. Use of a cationic group as the coating agent in a capillary channel (such as through a silylation agent) may improve accuracy of sample analysis.

In an exemplary embodiment of the invention, a compound comprising an anionic group and a reactive group may be used to coat the inner surface of a capillary channel. A capillary channel made of, for example, glass or fused silica may be coated with a compound containing an anionic group and at least one of silicon (e.g., a silylation agent), titanium, and zirconium. In an exemplary embodiment, the coating agent for a capillary channel may be a silylation agent having at least one anionic group. The anionic group may be, but is not limited to, a sulfate group, a carboxylate group, a sulfonate group or a phosphate group. In an exemplary embodiment, the anionic group is a chaotropic anionic group. Use of an anionic group as the coating agent in a capillary channel (such as through a silylation agent) may improve accuracy of sample analysis.

In an exemplary embodiment, the silylation agent is obtained by substituting the silicon atom by titanium or zirconium. One silylation agent may be used alone or two or more silylation agents may be used in combination. Examples of silylation agents include, but are not limited to, 2-(4-chlorosulfonylphenyl)ethyltrimethoxysilane and 2-(4-chlorosulfonylphenyl)ethyltrichlorosilane.

In an exemplary embodiment, the anionic group is attached to the inner wall of the capillary channel to form an anionic layer using the silylation agent as follows. First, a silylation agent is dissolved or dispersed in an organic solvent and thereby a treatment liquid is prepared. In an exemplary embodiment, the organic solvent to be used for preparing the treatment liquid is dichloromethane or toluene. The concentration of the silylation agent of the treatment liquid is not particularly limited. In an exemplary embodiment, the treatment liquid is passed through a capillary channel made of glass or molten silica and is heated. This heating allows the silylation agent to be bonded to the inner wall of the capillary channel by a covalent bond. As a result, the anionic group is affixed on the inner wall of the capillary channel. Thereafter, the inner wall is optionally washed with at least one of an organic solvent (e.g., but not limited to, dichloromethane, methanol, or acetone), an acid solution (e.g., but not limited to, phosphoric acid), an alkaline solution, and a surfactant solution (after treatment). Cationic groups may be attached to the inner wall of a capillary channel in the same manner described above for anionic groups.

Other coating agents that may be used to coat a capillary channel include compounds that are analogous to silylation agents having a cationic group or anionic group where titanium or zirconium atoms are substituted for the silicon atoms. Thus, in an exemplary embodiment, the capillary channel may be coated with a coating agent comprising at least one of silicon, titanium, and zirconium. In exemplary embodiments, a single silylation agent having a cationic or anionic group may be used, while two or more of such silylation agents may be used in combination in other embodiments.

In an exemplary embodiment, the inner wall of the capillary channel may be coated using a silylation agent by first preparing a treatment solution by dissolving or dispersing the silylation agent in an organic solvent (i.e., dichloromethane, or toluene, among others known in the art). The concentration of a silylation agent in a treatment solution is not particularly limited. A treatment solution may be passed through a capillary channel made of glass or fused silica, and heated in certain aspects of the present invention. As a result of heating, the silylation agent becomes covalently-bonded to the inner wall of the capillary channel, and a cationic or anionic group is arranged along the inner wall. After the heating step, the inner wall of the capillary channel may optionally be washed with at least one of an organic solvent (i.e., dichloromethane, methanol, or acetone, among others), an acid solution (i.e., phosphoric acid solution, among others), an alkaline solution, and a surfactant solution, among others.

In an exemplary embodiment of the invention, the capillary channel includes an “A” layer that is coated on an inner wall of the channel and a “B” layer that is optionally coated on the “A” layer. The “A” layer is affixed firmly to the inner wall of the capillary channel, such that once it forms, it is not easily detached, even when being washed, which allows the capillary channel so coated to be used repeatedly. Accordingly, from a practical approach, once the “A” layer is formed, it is not necessary to form the “A” layer each time an analysis is carried out, thereby increasing the ease and cost-effectiveness of the analysis.

In various exemplary embodiments, the “A” layer is a spacer layer formed of at least one selected from the group consisting of a polycationic polymer, a nonpolar polymer and a cationic group-containing compound. Attachment to the inner wall of the capillary channel may, for example, occur by physical adsorption and/or by covalent bond. When the layer includes polydiallyldimethylammonium chloride as a representative polycationic polymer, coating appears to occur by physical (i.e., non-covalent) adsorption. When the layer includes at least one of a nonpolar polymer and a cationic group-containing compound, coating appears to occur by covalent bond.

In various embodiments, the “B” layer is an anionic layer formed of an anionic group-containing compound.

The use of a capillary channel containing a “B” layer that is formed on an “A” layer where the “A” layer is affixed to an inner wall of the channel can prevent, for example, a protein in a blood sample, such as hemoglobin, from being adsorbed by the inner wall of the capillary channel. This lack of adsorption makes it possible to generate and maintain a good electroosmotic flow.

In the particular embodiment where the capillary channel is made of glass or fused silica and polydiallyldimethylammonium chloride is added, the polydiallyldimethylammonium chloride adsorbs firmly to the inner wall of the capillary channel, thereby forming an “A” layer. The “A” layer is not detached easily even when being washed. In an exemplary embodiment, the concentration of the polydiallyldimethylammonium chloride solution is in the range of about 1 to about 20 wt %, such as in the range of about 5 to about 10 wt %. In an exemplary embodiment, an alkaline solution is passed through the capillary channel followed by distilled water to wash the inner walls before the polydiallyldimethylammonium chloride solution is passed therethrough. In a particular embodiment, the alkaline solution is aqueous sodium hydroxide. After the polydiallyldimethylammonium chloride solution is passed through the capillary channel, distilled water is optionally passed through the capillary channel in order to remove any residual polydiallyldimethylammonium chloride that was not involved in formation of the “A” layer.

In an exemplary embodiment, a nonpolar polymer forms the “A” layer on the inner wall of the capillary channel. A suitable nonpolar polymer is a silicone polymer. The “A” layer may be formed by passing a solution containing the silicone polymer through the capillary channel. In the case where the capillary channel is made of glass or fused silica, the silicone polymer becomes fixed firmly to the inner wall of the capillary channel through formation of covalent bonds and thereby the “A” layer is generated. Such an “A” layer is not detached easily even when being washed.

Examples of suitable silicone polymers include, but are not limited to, polysiloxanes and polysilazanes. Exemplary polysiloxanes and the polysilazanes include polydiorganosiloxanes, polydiorganosilazanes and polyorganohydrosiloxanes. Specific examples of polysiloxanes and polysilazanes include polydialkylsiloxane, polydialkylsilazane, polyarylsiloxane, polyarylsilazane, polyalkylarylsiloxane, polydiarylsiloxane, cyclic siloxane and cyclic silazane.

The solution containing the silicone polymer is, for example, a solution containing the silicone polymer dispersed in a solvent, or a solution containing the silicone polymer dissolved in a solvent. After the dispersed solution or the dissolved solution of the silicone polymer is passed through the capillary channel, and the solvent is subsequently evaporatively removed by drying, a film layer of the silicone polymer is formed on the inner wall of the capillary channel. When heated, the silicone polymer forms a covalent bond to the inner wall of the capillary channel made of glass or fused silica. An exemplary heating treatment is carried out as follows: first, inert gas is passed through the capillary channel, in which the film layer of the silicone polymer has formed, to remove oxygen. Both ends of the capillary channel are then sealed by heating or the like. When the capillary channel in this state is heated, for example, at about 200 to about 450° C. for 10 minutes to 12 hours, the silicone polymer covalently bonds to the inner wall of the capillary channel. Subsequently, the capillary channel is cooled and both ends thereof are opened by cutting or the like. Unreacted silicone polymer is then removed by washing with a solvent. In this manner, an “A” layer made of the silicone polymer is formed on the inner wall of the capillary channel. The “A” layer made of the silicone polymer has a thickness, for example, in the range of about 50 to about 400 nm, such as in the range of about 100 to about 400 nm. A commercial product may be used as the capillary channel that includes the “A” layer formed of the silicone polymer.

When the “A” layer is formed on the inner wall of the capillary channel with a cationic group-containing compound, a compound containing the cationic group and a reaction group may be used. In a case where the capillary channel is made of glass or fused silica, a compound (e.g., a silylation agent) including the cationic group may be used. In an exemplary embodiment, the cationic group is an amino group or an ammonium group. An example of the cationic group-containing compound includes a silylation agent that contains at least one of the cationic groups of an amino group and an ammonium group. The amino group may be a primary amino group, a secondary amino group or a tertiary amino group.

The “A” layer may be formed in an exemplary procedure using a cationic group-containing silylation agent as follows: first, the silylation agent is dissolved or dispersed in an organic solvent and thereby a treatment liquid is prepared. An exemplary organic solvent suitable for preparing the treatment liquid is dichloromethane or toluene. The concentration of the silylation agent in the treatment liquid is not particularly limited. This treatment liquid is passed through a capillary channel made of glass or fused silica and heated. This heating allows the silylation agent to be bonded to the inner wall of the capillary channel through a covalent bond. As a result, the cationic group is placed on the inner wall of the capillary channel. Thereafter, the capillary channel is washed with at least one of an organic solvent (e.g., dichloromethane, methanol, or acetone), an acid solution (e.g., phosphoric acid), an alkaline solution, and a surfactant solution (after treatment). Although this washing is optional, it is preferred. A commercial product may be used as the capillary channel that includes the “A” layer formed of the silylation agent.

In an exemplary embodiment, the “B” layer is formed on the above-described “A” layer. The “B” layer may be formed by contacting the “A” layer with a solution containing an anionic group-containing compound as described herein. In this case, a solution for forming the “B” layer may be prepared separately. From a view of operation efficiency, however, it is preferable that a running buffer containing the anionic group-containing compound is prepared and passed through the capillary channel containing the “A” layer. In an exemplary embodiment, the anionic group of the anionic group-containing compound is a chaotropic anionic group with the result that a “B” layer is formed of a chaotropic anion.

The space required for instrumentation to analyze hemoglobin in a sample may be reduced by employing a microchip. A capillary channel that is part of a microchip is not particularly limited. The microchip may have a capillary channel formed by digging a groove on a microchip substrate, or a capillary channel may be buried in a groove on a microchip substrate.

The shape of the cross-section of a capillary channel formed by digging a groove on the substrate is not particularly limited. In exemplary embodiments, the cross-section of the capillary channel may be semicircular, or it may have an angular shape (e.g., a square-shaped cross-section). The inner wall of the capillary channel that is part of a microchip may or may not be coated as described above.

A microchip substrate into which a groove has been cut to form a capillary channel is not particularly limited. In various exemplary embodiments of the invention, the microchip substrate may comprise glass, fused silica, or a polymer (e.g., a plastic). For example, a glass microchip substrate may be a synthetic silica glass or a borosilicate glass. A polymer microchip substrate may be selected from those known in the art and includes, but is not limited to, polymethylmethacrylate (PMMA), cycloolefin polymer (COP), polycarbonate (PC), polydimethylsiloxane (PDMS), polystyrene (PS), polylactic acid, polyethylene (PE), polytetrafluoroethylene (PTFE) or polyetheretherketone (PEEK).

A capillary channel that is buried in a groove on a microchip may be made from the same substrates described herein. Also, the inner wall of a capillary channel buried in a groove formed on a microchip may be coated in the same manner described herein.

In an exemplary embodiment, the maximum inner diameter of a capillary channel in a microchip is between about 10 μm and about 200 μm, such as between about 25 μm and about 100 μm. In particular embodiments, the cross-sectional shape of a capillary channel in a micro-chip is not a circle, and the maximum inner diameter is the diameter of a circle whose area corresponds to the cross-sectional area of the region of the capillary channel that has a maximal cross-sectional area.

In an exemplary embodiment, the maximum length of a capillary channel in a microchip is less than about 15 cm, less than about 10 cm, less than about 5 cm, between about 2 cm and about 3 cm, between about 0.1 cm and about 15 cm, or between about 0.5 cm and about 15 cm. In an exemplary embodiment, the effective length of a capillary channel in a microchip is less than about 15 cm, less than about 10 cm, less than about 5 cm, between about 2 cm and about 3 cm, between about 0.1 cm and about 15 cm, or between about 0.5 cm and about 15 cm.

In an exemplary embodiment of the invention, a microchip having a capillary channel has a sample introduction channel that forms a cross shape with the capillary channel. The sample introduction channel and the CE channel may be filled with a buffer solution to which a chaotropic ion may be added. In a particular embodiment, the sample for analysis contains hemoglobin. The hemoglobin-containing sample may be introduced into a tank formed at one end of the sample introduction channel, and a voltage of between about 0.5 kV and about 10 kV may be applied to the sample introduction channel. By applying this voltage, the hemoglobin-containing sample may be transferred to the cross portion (e.g., where the sample introduction channel intersects with the CE channel). When a voltage of between about 0.5 kV and about 10 kV is applied to the CE channel, the hemoglobin in the sample moves toward a collection tank at one end of the CE channel. The difference in the rates of movement of different types of hemoglobin separated during electrophoresis may be determined using a detector.

The sample (also referred to as “sample to be analyzed”) is not particularly limited. In a particular embodiment of the invention, a sample comprises hemoglobin or is thought to comprise hemoglobin. The hemoglobin-containing sample may include blood or products containing hemoglobin that are commercially-available. A hemocyte-containing material, such as whole blood, may be hemolyzed to prepare a sample for CE. The hemolysis methods used on a hemocyte-containing material are not particularly limited and include ultrasonic treatments, freeze-thaw treatments, pressure treatments, osmotic pressure treatments and surfactant treatments. A hemolysate may be diluted (for example, with a solvent) to prepare the sample for analysis using CE methods of the present invention. The solvent used for dilution of a hemolysate is not particularly limited. In exemplary embodiments of the invention, a hemolysate may be diluted with water, normal saline solution or a buffer solution. In exemplary embodiments of the invention, a compound containing an anionic group may be added to the sample. The anionic group may be a chaotropic anionic group. As an example, a chaotropic anion, may be added at the time of hemolysis and/or at the time a hemolysate is diluted. A solvent used for dilution may comprise at least one chaotropic anion.

In a particular embodiment, the sample contains hemoglobin. The hemoglobin to be analyzed is not particularly limited. Examples thereof include normal hemoglobin, glycated hemoglobin (e.g., HbA1c, unstable HbA1c and GHbLys), and a genetic variation of hemoglobin.

In a particular embodiment of the invention, the sample to be analyzed comprises at least one of glycated hemoglobin (i.e., HbA1c, among others) sickle cell hemoglobin (HbS), hemoglobin C (HbC), hemoglobin M (HbM or membrane-attached hemoglobin), hemoglobin H (HbH), hemoglobin F (HbF or fetal hemoglobin), and modified Hb, among others. For example, a sample may comprise stable HbA1c and/or unstable HbA1c. In a particular embodiment, a sample to be analyzed comprises at least one modified Hb, such as a carbamoylated Hb or an acetylated Hb, among others. In a particular embodiment, stable HbA1c may be separated from other types of hemoglobin and detected. Similarly, hemoglobin types other than HbA1c may be separated and detected using the methods of the invention.

In exemplary embodiments, a hemoglobin-containing sample may comprise normal hemoglobin (HbA0); glycated hemoglobins (i.e., HbA1a, HbA1b, stable HbA1c, unstable HbA1c and GHbLys, among others); modified hemoglobins (i.e., carbamoylated Hb and acetylated Hb, among others); genetic variants of hemoglobin (i.e., HbS, HbC, HbM and HbH, among others); or fetal hemoglobin (HbF); among others. In a particular embodiment, stable HbA1c may be separated and detected, and other types of hemoglobin in the sample may be separated from and analyzed simultaneously with the stable HbA1c.

As used herein, the term “buffer” or “running buffer” denotes a buffer solution (buffer) that is used in a separation process. In an exemplary embodiment, a sample is added to a running buffer containing an anionic group-containing compound, and a voltage is then applied to both ends of a capillary channel to electrophorese a complex of the sample and an anionic group-containing compound.

The running buffer is not particularly limited. In an exemplary embodiment, the buffer contains acid. Examples of suitable acids include, but are not limited to, maleic acid, tartaric acid, succinic acid, fumaric acid, phthalic acid, malonic acid, and malic acid. In an exemplary embodiment, the running buffer contains a weak base. Examples of suitable weak bases include, but are not limited to, arginine, lysine, histidine, and tris. The running buffer typically has a pH, for example, in the range of 4.5 to 6. The types of buffer in the running buffer include, but are not limited to, morpholinoethanesulfonic acid (MES), N-(2-acetamido)iminodiacetic acid (ADA), N-(2-acetamido)-2-aminoethanesulfonic acid (ACES), N,N-bis(2-hydroxyethyl)-2-aminoethanesulfonic acid (BES), 3-morpholinopropanesulfonic acid (MOPS), N-tris(hydroxymethyl)methyl-2-aminoethanesulfonic acid (TES) and 2-[4-(2-hydroxyethyl)-1-piperazinyl]ethanesulfonic acid (HEPES).

In an exemplary embodiment of the invention, the CE buffer solution and/or a hemoglobin-containing sample comprises one or more compounds containing an anionic group. In an exemplary embodiment, the anionic group is a chaotropic anionic. For instance, the buffer may contain a chaotropic anion at a concentration between about 10 mmol/L and about 50 mmol/L.

In an exemplary embodiment of the invention, the CE buffer solution and/or the hemoglobin-containing sample comprises at least one anionic group-containing a compound at a concentration between about 0.01% and about 5% by weight (wt %).

In an exemplary embodiment of the invention, an anionic group-containing compound is added to the electrophoresis buffer solution that is used during CE of a sample containing hemoglobin to separate stable HbA1c, unstable HbA1c, and/or modified Hb. Not wishing to be bound by theory, it appears that the anionic group-containing compound complexes with each of the stable HbA1c and unstable HbA1c via ionic and/or hydrophobic interactions. The charge states of the stable HbA1c and the unstable HbA1c are different from each other. When each type of HbA1c is complexed with the anionic group-containing compound, the complex is negatively charged as a whole. A chaotropic anion may be present in the capillary channel in, for example, the buffer solution or in the sample. As discussed herein, addition of a chaotropic ion was discovered to improve the water solubility of hydrophobic molecules. Therefore, in the presence of a chaotropic ion, the hydrophobic interactions of the complex are weakened, and the charge state of the stable HbA1c or unstable HbA1c has a significant effect on the charge state of the complex. As a result, the difference of the charge state between the stable HbA1c complex and the unstable HbA1c complex is greater than the difference in the charge states of the uncomplexed stable HbA1c and the unstable HbA1c, and it is believed that it is this larger difference in the charge states that permits their successful separation using CE. It may be that the same mechanism permits the separation of the stable HbA1c from the modified Hb.

In an exemplary embodiment, an anionic group-containing compound forms a complex together with the sample. In a particular embodiment, the anionic group-containing compound is an anionic group-containing polysaccharide. Examples of the anionic group-containing polysaccharide include, but are not limited to, a sulfated polysaccharide, a carboxylated polysaccharide, a sulfonated polysaccharide and a phosphorylated polysaccharide. In a particular embodiment, the polysaccharide is a sulfated polysaccharide or a carboxylated polysaccharide. Examples of the sulfated polysaccharide include, but are not limited to, chondroitin sulfate or heparin. In a particular embodiment, the sulfated polysaccharide is chondroitin sulfate. An example of the carboxylated polysaccharide is alginic acid or a salt thereof (for instance, sodium alginate). Examples of chondroitin sulfates include, but are not limited to, chondroitin sulfate A, chondroitin sulfate B, chondroitin sulfate C, chondroitin sulfate D, chondroitin sulfate E, chondroitin sulfate H and chondroitin sulfate K.

In exemplary embodiments of the invention, the hemoglobin in a sample may be electrophoresed in the presence of both at least one chaotropic anion and at least one anionic group-containing compound. Not wishing to be bound by theory, it appears that when an anionic group-containing compound is present during CE of a sample containing hemoglobin, the hemoglobin in the sample forms a complex with the anionic group-containing compound. Electrophoresis in the presence of at least one chaotropic anion and at least one anionic group-containing compound may further improve analysis accuracy and reduce analysis time of hemoglobin-containing samples. The length of the capillary channel may be shortened if analysis accuracy is increased.

In exemplary embodiments of the invention, the anionic group-containing compound may be added to a sample prior to its application to a capillary channel or it may be added to a buffer solution in the capillary channel to which a sample is applied. The anionic group-containing compound may also be added directly to the sample or to a solvent used for diluting a hemoglobin-containing specimen (i.e., hemolysate, among others). In a particular embodiment, a buffer solution that is used to fill up the capillary channel contains at least one anionic group-containing compound.

In an exemplary embodiment, the anionic group-containing compound is used to form the “B” layer described herein.

The concentration of an anionic group-containing compound in the sample, a dilution solvent, and/or the CE buffer solution is not particularly limited. In an exemplary embodiment, the concentration is between about 0.01% and about 5% by weight, such as about 0.1% and about 2% by weight.

The location of the chaotropic anion during CE is not particularly limited. For example, the chaotropic anion may be present in the running buffer and/or in the sample and/or as a layer on the inner wall of the capillary channel. In an exemplary embodiment, the anionic group-containing compound is a chaotropic anion. The chaotropic anion itself is not particularly limited. Chaotropic anions include, but are not limited to, perchlorate ions (ClO₄⁻), thiocyanate ions (SCN⁻), trichloroacetate ions (CCl₃COO⁻), trifluoroacetate ions (CF₃COO⁻), nitrate ions (NO₃⁻), dichloroacetate (CCl₂COO⁻), and halogenide ions. The chaotropic anion may be added to the sample and/or the running buffer solution in the acid form. For example, trifluoroacetic acid may be added to the sample and/or the running buffer solution. More than one chaotropic anion may be present during electrophoresis. Halogenide ions that may be used in certain aspects of the present invention are not particularly limited. Specific halogenide ions include fluoride ions (F⁻), chloride ions (Cl⁻), bromide ions (Br⁻), iodide ions (I⁻) and astatide ions (At⁻). In a particular embodiment of the invention, the halogenide ions are bromide ions (Br⁻) and/or iodide ions (I⁻).

In an exemplary embodiment of the invention, the CE buffer solution and/or the sample comprises an anion such as a chaotropic anion. In another exemplary embodiment, the CE buffer solution and/or the sample comprises both a chaotropic anion and an anionic group-containing compound, wherein the chaotropic anion and the anionic group-containing compound are different. In an exemplary embodiment, the anionic group-containing compound is an anionic group-containing polysaccharide. In a particular embodiment, the anionic group-containing polysaccharide is a chondroitin sulfate.

In a particular embodiment of the invention, the sample contains hemoglobin and is electrophoresed in the presence of at least one chaotropic anion.

In exemplary embodiments of the present invention, the chaotropic anion may be added to the sample (e.g., prior to the introduction of the sample into the capillary channel) and/or the chaotropic anion may be added to the running buffer solution in the capillary channel. For example, the chaotropic anion may be added directly to the sample just prior to addition of the sample to the capillary channel or it may be added to a solution that is used to dilute the sample. In a particular embodiment, a electrophoresis buffer solution containing the chaotropic anion is used to fill the capillary channel prior to application of the sample.

Not wishing to be bound by theory, it appears that a chaotropic anion enhances solubility of a hydrophobic molecule in water by disrupting interactions between water molecules and inhibiting a decrease in the entropy of water caused by contact with a hydrophobic molecule.

The chaotropic anion may be added to the sample, to the buffer solution or to both the sample and the buffer solution. The chaotropic anion may be introduced as a salt (e.g., guanidinium chloride or lithium perchlorate) or as a compound that generates the chaotropic anion following ionization (e.g., trichloroacetic acid, thiocyanic acid, perchloric acid, among others). The chaotropic anion may be generated in the sample and/or the buffer solution, when the salt or ion-generating compound is dissolved. The chaotropic anion may be part of an acid salt, a neutral salt, or a basic salt. The salts or other compound that generate a chaotropic anion are not particularly limited. In exemplary embodiments of the invention, the chaotropic anion may be introduced as an alkali metal halide (e.g., potassium iodide or sodium bromide), an alkaline earth halide (e.g., calcium bromide or magnesium iodide) or as a free acid (e.g., perchloric acid, thiocyanic acid, trichloroacetic acid or trifluoroacetic acid). Addition of a chaotropic anion to the sample and/or the CE buffer solution may be accomplished by adding a salt containing the chaotropic anion or a compound that generates the chaotropic anion by ionization to the sample and/or the CE buffer solution.

The concentration of the chaotropic anion in a sample and/or a CE buffer solution at the time of electrophoresis is not particularly limited. In exemplary embodiments of the invention, a sample and/or a CE buffer solution comprises at least one chaotropic anion at a concentration between about 1 mmol/L and about 3000 mmol/L, such as between about 5 mmol/L and about 100 mmol/L, such as between about 10 mmol/L and about 50 mmol/L at the time of electrophoresis.

In an exemplary embodiment, a sample is introduced into a running buffer containing an anionic group-containing compound, and voltage is then applied across both ends of the capillary channel to perform electrophoretic separation of a complex of the sample and the anionic group-containing compound. In an exemplary embodiment, the anionic group of the anion group-containing compound is a chaotropic anion.

In an exemplary embodiment, CE is carried out using a sample containing hemoglobin as follows: first, a capillary channel made of glass or molten silica is prepared. An anionic group-containing compound is affixed to the inner wall of the capillary channel by a covalent bond. Purified water is subsequently passed through the capillary channel as a wash for example, about 1 to about 10 minutes, and at a pressure of, for example, about 0.05 to about 0.1 MPa. Subsequently, a running buffer containing an anionic group-containing polysaccharide such as chondroitin sulfate is passed through the capillary channel for example, about 10 to about 60 minutes and at a pressure of, for example, about 0.05 to about 0.1 MPa. With the capillary channel filled with the running buffer, a hemoglobin-containing sample is introduced into the capillary channel, and voltage is then applied to both the ends of the capillary channel to carry out electrophoresis. The hemoglobin-containing sample is not particularly limited and is, for example, a sample obtained by hemolyzing whole blood. This sample may optionally be diluted with purified water or a running buffer. The hemoglobin-containing sample is introduced from the anode side of the capillary channel. The hemoglobin thus introduced forms a complex by bonding with the anionic group-containing polysaccharide contained in the running buffer. Voltage application generates an electroosmotic flow in the running buffer contained in the capillary channel and thereby the complex is transferred toward the cathode side of the capillary channel. The voltage applied is, for example, in the order of about 10 to about 30 kV. This transfer is detected by an optical method that is not particularly limited. Preferably, detection is carried out with a wavelength of about 415 nm.

In various exemplary embodiments using the apparatuses shown as figures in the application, purified water is passed through the capillary channel 3x for sample analysis and the capillary channel 3y for sample introduction to wash them. The time for which the purified water is passed therethrough and the pressure applied when it is passed therethrough are, for example, as described herein. Subsequently, a running buffer containing an anionic group-containing polysaccharide such as, for example, chondroitin sulfate is passed through the capillary channel 3x for sample analysis and the capillary channel 3y for sample introduction under pressure applied with, for example, a pump. The time for which it is passed therethrough and the pressure thereof are, for example, as described herein. Thereafter, the capillary channel 3x for sample analysis and the capillary channel 3y for sample introduction are filled with the running buffer by pressure or capillary action.

In an exemplary embodiment, when the microchip electrophoresis apparatus is not in use (i.e., when no analysis is carried out), the step of filling the capillary channels with the running buffer is completed beforehand, since it makes it possible to omit the respective steps described above and to proceed directly to the step of sample introduction.

A hemoglobin-containing sample is introduced into the second introduction tank 2c. Examples of the hemoglobin-containing sample are as described herein. When the microchip electrophoresis apparatus has the pretreatment tank (not shown in the figures), the hemoglobin-containing sample is introduced into the pretreatment tank and is pretreated there. Subsequently, voltage is applied to the electrode 6c and the electrode 6d to generate a potential difference between the ends of the capillary channel 3y for sample introduction. Thus, the hemoglobin-containing sample is introduced into the capillary channel 3y for sample introduction. The hemoglobin thus introduced is bonded with an anionic group-containing polysaccharide contained in the running buffer to form a complex. Voltage is applied to generate an electroosmotic flow in the running buffer contained in the capillary channel 3y for sample introduction and thereby the complex is transferred to the intersection part between the capillary channel 3x for sample analysis and the capillary channel 3y for sample introduction.

The potential difference between the electrode 6c and the electrode 6d is, for instance, in the range of about 0.5 to about 5 kV.

Next, voltage is applied to the electrode 6a and the electrode 6b to generate a potential difference between the ends of the capillary channel 3x for sample analysis. In this manner, the capillary channel having a potential difference between the ends thereof is changed momentarily from the capillary channel 3y for sample introduction to the capillary channel 3x for sample analysis, so that as shown with arrows in FIGS. 23 and 24, the sample 8 is transferred to the first recovery tank 2b side from the intersection part between the capillary channel 3x for sample analysis and the capillary channel 3y for sample introduction.

The potential difference between the electrode 6a and the electrode 6b is, for example, in the range of about 0.5 to about 5 kV.

Subsequently, the respective components of the hemoglobin-containing sample separated due to the difference in transfer rate are detected with the detector 7. Thus, the respective components of the hemoglobin-containing sample can be separated for individual analysis.

Once a capillary channel is filled with an electrophoresis buffer solution, a sample may be introduced into the buffer solution, and voltage may be applied to both ends of the capillary channel to carry out electrophoresis. The sample, which in a particular embodiment contains hemoglobin, may be introduced from the anode side of the capillary channel. Application of voltage generates an electroosmotic flow in the electrophoresis buffer solution in the capillary channel and hemoglobin in the applied sample moves toward the cathode end of the capillary channel. In certain aspects of the present invention where an anionic group-containing compound is present during electrophoresis, hemoglobin moves toward the cathode end of the capillary channel as part of a complex comprising the hemoglobin and the anionic group-containing compound. The voltage applied to the capillary channel during electrophoresis is sufficient to permit separation of at least one type of hemoglobin in a sample, and may be between about 1 kV and about 30 kV. In exemplary embodiments, the CE may be carried out at a temperature between about 1° C. and about 60° C., between about 5° C. and about 35° C., about 20° C., and about room temperature. The electrophoresed hemoglobin may be detected using methods known in art, such as, for example, an optical method or a fluorescence method. The optical method used for detection of hemoglobin in the present invention is not particularly limited and may be performed by measuring absorbance at, for example, a wavelength of between about 400 nm and about 600 nm, or between about 400 nm and about 450 nm, and in certain aspects at a wavelength of about 415 nm and/or at about 550 nm.

Analysis of a sample using a capillary channel that includes an “A” layer formed of polydiallyldimethylammonium chloride is described in an exemplary embodiment of the invention. First, a capillary channel made of glass or fused silica is prepared. Next, an alkaline solution such as an aqueous sodium hydroxide is passed through the capillary channel under pressure applied by, for example, a pump. Subsequently, distilled water is passed through the capillary channel as a wash. The time for which each of the alkaline solution and the distilled water is passed therethrough is, for example, about 1 to about 10 minutes, and the pressure when each of the alkaline solution and the distilled water is passed therethrough is, for example, about 0.05 to about 0.1 MPa. Next, a polydiallyldimethylammonium chloride solution is passed through the capillary channel under pressure applied by, for example, a pump. The time for which the polydiallyldimethylammonium chloride solution is passed therethrough is, for example, about 5 to about 30 minutes, and the pressure when the polydiallyldimethylammonium chloride solution is passed therethrough is, for example, about 0.05 to about 0.1 MPa. The distilled water is then passed through the capillary channel under pressure applied by, for example, a pump to remove residual polydiallyldimethylammonium chloride. The time for which the distilled water is passed therethrough and the pressure when the distilled water is passed therethrough is as same as in the case of the aforementioned washing. In this manner, the polycationic “A” layer made of the polydiallyldimethylammonium chloride is formed on the inner wall of the capillary channel. In this state, the time and the pressure are determined suitably according to an inner diameter and a length of the capillary channel. Each time and pressure range mentioned above is an example which is suitable for the capillary channel that has an inner diameter of about 50 μm and a length of about 320 mm. The same applies to the following.

Next, a running buffer containing an anionic group-containing compound, such as chondroitin sulfate, is passed through the capillary channel under pressure applied by, for example, a pump. The time for which the running buffer is passed therethrough is, for example, about 10 to about 60 minutes, and the pressure when the running buffer is passed therethrough is, for example, about 0.05 to about 0.1 MPa. As a result, a “B” layer formed of chondroitin sulfate is coated on the “A” layer. In this state, a hemoglobin-containing sample is introduced into the capillary channel, and voltage then is applied across both ends of the capillary channel to carry out electrophoresis. The hemoglobin-containing sample is not particularly limited and is, for example, a sample obtained by hemolyzing whole blood. This sample may optionally be diluted with distilled water or a running buffer. The hemoglobin-containing sample is introduced from the anode side of the capillary channel. The hemoglobin thus introduced forms a complex by being bonded with the anionic group-containing compound contained in the running buffer. The applied voltage generates an electroosmotic flow in the running buffer contained in the capillary channel and thereby the complex is transferred toward the cathode side of the capillary channel. The voltage applied is, for example, in the order of 5 to 30 kV. This transfer is detected by an optical method. The detection made by the optical method is not particularly limited. Preferably, it is carried out at a wavelength of about 415 nm.

CE using a capillary channel that includes an “A” layer formed of at least one of a suitable nonpolar polymer and/or a suitable cationic group-containing compound can be carried out in the same manner as described above except for the preparation of the “A” layer.

CE using a capillary channel that includes an “A” layer formed of the polycationic polydiallyldimethylammonium chloride using the apparatuses shown as figures in the application (with four capillary channels) is described in an exemplary embodiment of the invention. First, an alkaline solution, such as an aqueous sodium hydroxide, is passed through the capillary channel 3x for sample analysis and the capillary channel 3y for sample introduction under pressure applied by, for example, a pump. Subsequently, distilled water is passed through the capillary channel 3x for sample analysis and the capillary channel 3y for sample introduction to wash them. The time for which each of the alkaline solution and the distilled water is passed therethrough and the pressure applied when each of them is passed therethrough are, for example, as described above. Next, the polydiallyldimethylammonium chloride solution is passed through the capillary channel 3x for sample analysis and the capillary channel 3y for sample introduction under pressure applied by, for example, a pump. The time for which it is passed therethrough and the pressure thereof are, for example, as described above. Then, distilled water is passed through the capillary channel 3x for sample analysis and the capillary channel 3y for sample introduction under pressure applied by, for example, a pump to remove residual polydiallyldimethylammonium chloride. The time for which it is passed therethrough and the pressure thereof are, for example, as described above. In this manner, the “A” layer is formed on the inner wall of the capillary channel 3x for sample analysis and the capillary channel 3y for sample introduction with the polydiallyldimethylammonium chloride.

Next, a running buffer containing an anionic group-containing polysaccharide, such as chondroitin sulfate, is passed through the capillary channel 3x for sample analysis and the capillary channel 3y for sample introduction under pressure applied by, for example, a pump. The time for which it is passed therethrough and the pressure thereof are, for example, as described above. Thereby, the “B” layer made of such as chondroitin sulfate is coated on the “A” layer. Thereafter, the capillary channel 3x for sample analysis and the capillary channel 3y for sample introduction are filled with the running buffer by pressure or capillary action.

It is preferable that when the microchip electrophoresis apparatus is not in use (when no analysis is carried out), the step of filling them with the running buffer be completed beforehand, since it makes it possible to omit the respective steps described above and to proceed directly to the following step.

Subsequently, a sample, which in a particular embodiment contains hemoglobin, is introduced into the second introduction tank 2c. Examples of the hemoglobin-containing sample are as described above. When the microchip electrophoresis apparatus has the pretreatment tank (not shown in the figures), the hemoglobin-containing sample is introduced into the pretreatment tank and is pretreated there. Subsequently, voltage is applied to the electrode 6c and the electrode 6d to generate a potential difference between the ends of the capillary channel 3y for sample introduction. Thus, the hemoglobin-containing sample is introduced into the capillary channel 3y for sample introduction. The hemoglobin thus introduced is bonded with an anionic group-containing polysaccharide contained in the running buffer to form a complex. Voltage is applied to generate an electroosmotic flow in the running buffer contained in the capillary channel 3y for sample introduction and thereby the complex is transferred to the intersection part between the capillary channel 3x for sample analysis and the capillary channel 3y for sample introduction.

The potential difference between the electrode 6c and the electrode 6d is, for instance, in the range of 0.5 to 5 kV.

Next, voltage is applied to the electrode 6a and the electrode 6b to generate a potential difference between the ends of the capillary channel 3x for sample analysis. In this manner, the capillary channel having a potential difference between the ends thereof is changed momentarily from the capillary channel 3y for sample introduction to the capillary channel 3x for sample analysis, so that as shown with arrows in FIGS. 23 and 24, the sample 8 is transferred to the first recovery tank 2b side from the intersection part between the capillary channel 3x for sample analysis and the capillary channel 3y for sample introduction.

The potential difference between the electrode 6a and the electrode 6b is, for example, in the range of 0.5 to 5 kV.

Subsequently, the respective components of the hemoglobin-containing sample separated due to the difference in transfer rate are detected with the detector 7. Thus, the respective components of the hemoglobin-containing sample can be separated to be analyzed.

CE using the aforementioned four capillary channels that includes an “A” layer formed of at least one of a suitable nonpolar polymer and a suitable cationic group-containing compound can be carried out in the same manner as described above except for the preparation of the “A” layer.

The methods of the present invention may be used to diagnose and monitor diseases. A sample of biological fluid or bodily fluid may be applied to the CE apparatus of the present invention for separation of the components of the fluid. The presence of a particular component of the fluid may indicate a particular disease. The amount of a particular component in a sample also may be determined after electrophoresis of the sample.

As used herein, the term “biological fluids of patients” refers to fluids from living organisms. The term encompasses “bodily fluids” which are found in the body of living organisms. Human bodily fluids may include prostatic fluid, seminal fluid, whole blood, serum, urine, breast biopsy fluid, gastrointestinal fluid, and vaginal fluid.

The present invention provides collecting human blood from a patient and assaying the blood to determine the level of glycated hemoglobin in the blood sample through the use of the CE technology and apparatus of the present invention to diagnose and monitor diabetes. For example, a blood sample may be collected and applied to the CE apparatus and separated into its components. The amount of glycosylated Hb is detected and analyzed for determining its percentage.

Glycosylated hemoglobin has been recommended for both checking blood sugar control in people who might be pre-diabetic and for monitoring blood sugar control in patients with more elevated levels, termed diabetes mellitus. The amount of glycosylated hemoglobin provides a way to monitor diabetes because it provides information as to whether a patient's diabetes is under control. As a reference, a non-diabetic or normal subject has less than 6% HbA1C in his blood, and a patient having less than 6% HbA1C in his blood indicates that his diabetes is under control.

The CE apparatus of the present invention may be used to diagnose and monitor diabetes in a patient.

An aspect of the invention is directed to a hemoglobin analysis kit comprising at least one CE buffer solution containing one or more anionic group containing compounds, such as compounds containing a chaotropic anion, and, optionally, a hemolysis solution, a solvent for diluting a hemolysate, a microchip having a CE channel or a combination thereof. In an exemplary embodiment of the invention, the kit comprises a CE buffer solution comprising at least one of a perchlorate ion, a thiocyanate ion, a trichloroacetate ion, a trifluoroacetate ion, an iodide ion, or a bromide ion, among other chaotropic anions. In particular embodiments, the kit comprises a CE buffer solution comprising at least one of a perchlorate ion and a thiocyanate ion. In an exemplary embodiment, the CE buffer solution in a kit may comprise at least one anionic group-containing compound (e.g., chondroitin sulfate). In an exemplary embodiment, the kit comprises a CE buffer solution comprising a chaotropic anion at a concentration between about 10 mmol/L and about 50 mmol/L, and, optionally, at least one anionic group-containing compound at a concentration between about 0.01 wt % and about 5 wt %. In a particular embodiment, the kit comprises a CE buffer solution and a hemolysis solution, wherein the hemolysis solution comprises at least one chaotropic anion. In another particular embodiment, the kit comprises a CE buffer solution, a hemolysis solution, and a hemolysate dilution solvent wherein the hemolysate dilution solvent comprises at least one chaotropic anion.

The kit may be a kit for diagnosing or monitoring diabetes. For example, the kit may be used to diagnose whether a subject may develop diabetes or may have diabetes. The kit may also be used to determine the level of glycated hemoglobin in a patient for monitoring the progression of diabetes.

Without further description, it is believed that one of ordinary skill in the art can, using the preceding description and the following illustrative examples, make and utilize the claimed invention. The following working examples therefore, specifically point out preferred embodiments of the present invention, and are not to be construed as limiting in any way the remainder of the disclosure. All articles, publications, patents and documents referred to throughout this application are hereby incorporated by reference in their entirety.

### EXAMPLES

The Examples 1 to 6 that follow disclose analysis methods in which stable HbA1c and unstable HbA1c are separated and detected. Example 2 discloses an analysis method in which stable HbA1c and carbamoylated Hb are separated and detected, and Example 3 discloses an analysis method in which stable HbA1c and acetylated Hb are separated and detected.

### Example 1

A hemoglobin-containing sample was prepared as follows. First, glucose was added to whole human blood at a concentration of 500 mg/100 mL, and incubated at 37° C. for 3 hours. After incubation, the reaction mixture was diluted fifteen fold with purified water to produce a hemoglobin-containing sample. Then, a capillary channel made of fused silica (overall length: 32 cm, effective length: 8.5 cm, and inner diameter: 50 μm) was prepared for electrophoresis. A buffer solution (pH 4.8) was prepared comprising a solution of 50 mmol/L fumaric acid-arginine acid with 0.8% by weight chondroitin sulfate C. Perchloric acid was added to this buffer solution to a concentration of 30 mmol/L. The buffer solution, to which the perchloric acid was added, was used to pressure fill the capillary channel at a pressure of 0.1 MPa (1000 mbar), and then the sample was injected into the anode side of the capillary channel. A 10 kV voltage was applied to both ends of the capillary channel to carry out electrophoresis, and hemoglobin was detected at an absorbance of 415 nm as it was electrophoresed. The effective length of the capillary channel was the length from the sample injection position at the anode side of the capillary channel to the point at which the absorbance was detected.

### Example 2

The analysis was performed as in Example 1 except that thiocyanic acid, instead of perchloric acid, was added to the buffer solution to a concentration of 30 mmol/L.

### Example 3

The analysis was performed as in Example 1 except that potassium iodide, instead of the perchloric acid, was added to the buffer solution to a concentration of 30 mmol/L.

### Example 4

The analysis was performed as in Example 1 except that potassium bromide, instead of perchloric acid, was added to the buffer solution to a concentration of 30 mmol/L.

### Example 5

The analysis was performed as in Example 1 except that trichloroacetic acid ion, instead of perchloric acid, was added to the buffer solution to a concentration of 30 mmol/L.

### Example 6

The analysis was performed as in Example 1 except that trifluoroacetic acid ion, instead of perchloric acid, was added to the buffer solution to a concentration of 30 mmol/L.

### Example 7

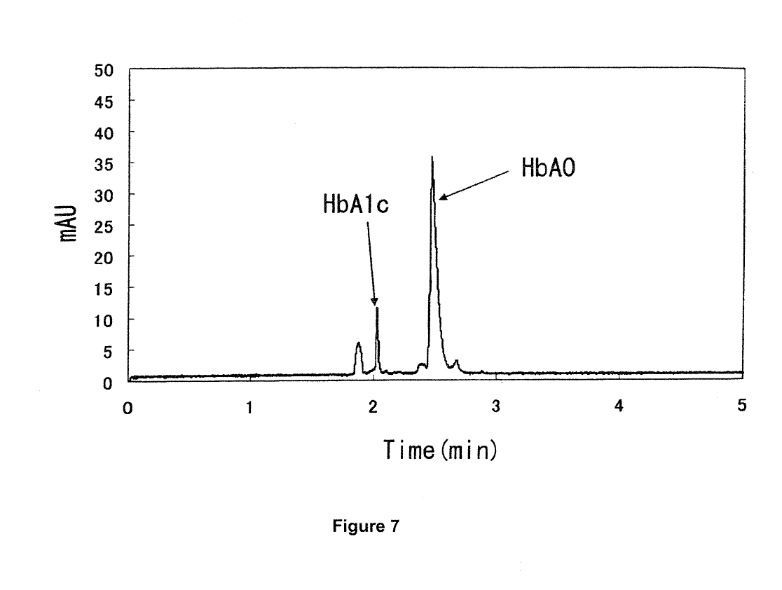

A capillary channel (with an overall length of 32 cm, an effective length of 8.5 cm, and an inner diameter of 50 μm) made of fused silica was prepared. An aqueous sodium hydroxide (1 mol/L) was passed through this capillary channel at a pressure of 0.1 MPa (1000 mbar) for 10 minutes. Subsequently, distilled water was passed through this capillary channel at the same pressure as described above for 20 minutes to wash it. Then, a polydiallyldimethylammonium chloride solution (10 wt %) was passed through the capillary channel at the same pressure as described above for 30 minutes. Subsequently, distilled water was passed through the capillary channel at the same pressure as described above for 20 minutes to form the A layer made of polydiallyldimethylammonium chloride on the inner wall of the capillary channel. Then, a running buffer (pH 5.5) was prepared that contains chondroitin sulfate added to 100 mM malic acid and an arginine acid aqueous solution at a ratio of 0.5 wt %. This running buffer was passed through the capillary channel, in which the A layer is formed, at the same pressure as described above, and thereby the B layer is formed on the A layer. With the capillary channel being filled with the running buffer, a sample containing hemoglobin dissolved in distilled water was injected into the capillary channel. Thereafter, a voltage of 10 kV was applied across both ends of the capillary channel, and thereby electrophoresis was carried out. The hemoglobin-containing sample was injected into the capillary channel from the anode side thereof. The hemoglobin that had been transferred was detected at an absorbance of 415 nm. This result is shown in the electropherogram in FIG. 7. As shown in FIG. 7, in this example, it was possible to detect normal hemoglobin (HbA0) and glycated hemoglobin (HbA1c) separately. Furthermore, as for the capillary channel used in this example, because the B layer was formed simply by passing through the running buffer therein after being washed, it was possible to carry out the analysis immediately.

### Example 8

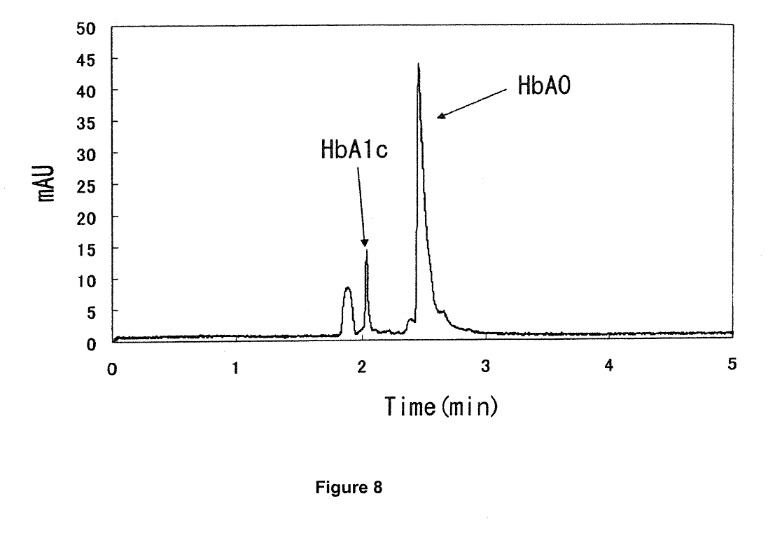

A capillary channel (with an overall length of 32 cm, an effective length of 8.5 cm, and an inner diameter of 50 μm) made of fused silica was prepared. The capillary channel had an A layer formed with a silylation agent having an amino group that was fixed to the inner wall thereof by a covalent bond. Distilled water was passed through this capillary channel at a pressure of 0.1 MPa (1000 mbar) for 20 minutes to wash it. Then, a running buffer (pH 5.5) was prepared that contains chondroitin sulfate added to 100 mM malic acid and an arginine acid aqueous solution at a ratio of 0.5 wt %. This running buffer was passed through the capillary channel at the same pressure as described above, and thereby the B layer was formed on the A layer. With the capillary channel being filled with the running buffer, a sample containing hemoglobin dissolved in distilled water was injected into the capillary channel. Thereafter, a voltage of 10 kV was applied across both ends of the capillary channel, and thereby electrophoresis was carried out. The hemoglobin-containing sample was injected into the capillary channel from the anode side thereof. The hemoglobin that had been transferred was detected at an absorbance of 415 nm. This result is shown in the electropherogram in FIG. 8. As shown in FIG. 8, in this example, it was possible to detect normal hemoglobin (HbA0) and glycated hemoglobin (HbA1c) separately. Furthermore, as for the capillary channel used in this example, because the B layer was formed simply by passing through the running buffer therein after being washed, it was possible to carry out the analysis immediately. In this state, the same analysis was carried out 10 times with the same sample as described above to evaluate precision. This result is shown in the following Table 1. In Table 1, a relative area (%) denotes a ratio (%) of each peak area of the normal hemoglobin (HbA0) and the glycated hemoglobin (HbA1c) relative to a total peak area. As shown in Table 1, a value of coefficient of variation (CV) is small in each of the normal hemoglobin (HbA0) and the glycated hemoglobin (HbA1c). Thereby, it can be said that the analytical processes of the present invention is excellent in the repeatability.

TABLE 1

Relative Area (%)

| No. | HbA1c | HbA0 |
|---|---|---|
| 1 | 10.08 | 89.92 |
| 2 | 10.37 | 89.63 |
| 3 | 10.18 | 89.82 |
| 4 | 10.49 | 89.51 |
| 5 | 10.34 | 89.66 |
| 6 | 10.30 | 89.70 |
| 7 | 9.89 | 90.11 |
| 8 | 10.17 | 89.83 |
| 9 | 10.24 | 89.76 |
| 10 | 10.32 | 89.68 |
| Average | 10.24 | 89.76 |
| Coefficient of | 1.7 | 0.2 |
| Variation (CV) | | |

### Example 9

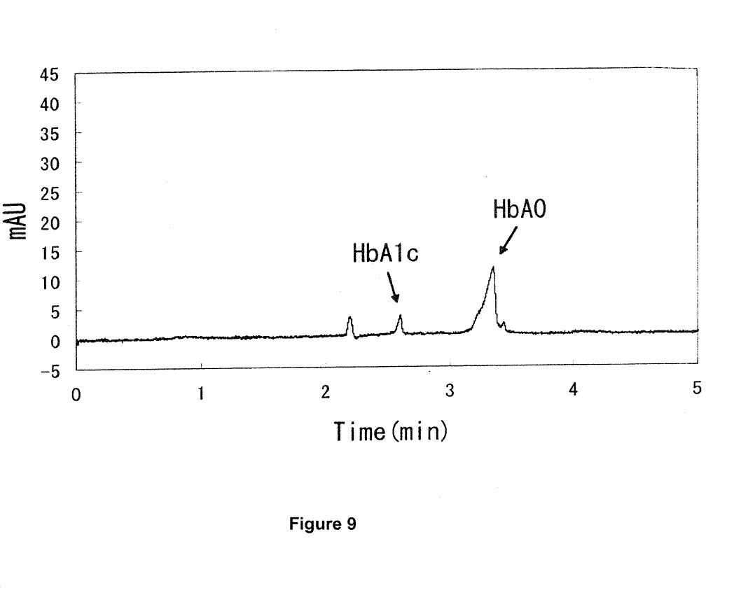

A capillary channel (with an overall length of 32 cm, an effective length of 8.5 cm, and an inner diameter of 50 μm) made of fused silica was prepared. The capillary channel had an A layer formed with a silylation agent having an amino group that was fixed to the inner wall thereof by a covalent bond. Distilled water was passed through this capillary channel at a pressure of 0.1 MPa (1000 mbar) for 20 minutes to wash it. Then, a running buffer (pH 5.5) was prepared that contains sodium alginate added to 100 mM malic acid and an arginine acid aqueous solution at a ratio of 0.8 wt %. This running buffer was passed through the capillary channel at the same pressure as described above, and thereby the B layer is formed on the A layer. With the capillary channel being filled with the running buffer, a sample containing hemoglobin dissolved in distilled water was injected into the capillary channel. Thereafter, a voltage of 10 kV was applied across both ends of the capillary channel, and thereby electrophoresis was carried out. The hemoglobin-containing sample was injected into the capillary channel from the anode side thereof. The hemoglobin that had been transferred was detected at an absorbance of 415 nm. This result is shown in the electropherogram in FIG. 9. As shown in FIG. 9, in this example, it was possible to detect normal hemoglobin (HbA0) and glycated hemoglobin (HbA1c) separately. Furthermore, as for the capillary channel used in this example, because the B layer was formed simply by passing through the running buffer therein after being washed, it was possible to carry out the analysis immediately.

### Example 10

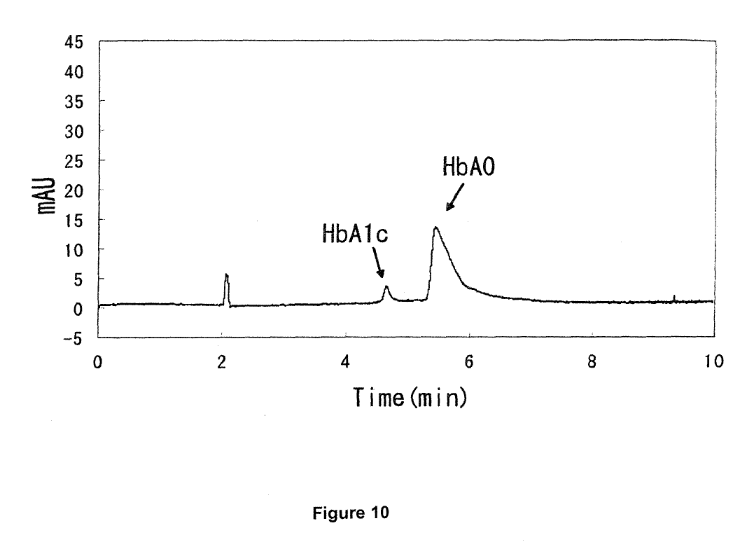

A capillary channel (with an overall length of 32 cm, an effective length of 8.5 cm, and an inner diameter of 50 μm) made of fused silica was prepared. The capillary channel had an A layer formed with a silylation agent having an amino group that was fixed to the inner wall thereof by a covalent bond. Distilled water was passed through this capillary channel at a pressure of 0.1 MPa (1000 mbar) for 20 minutes to wash it. Then, a running buffer (pH 5.5) was prepared that contains heparin sodium added to 100 mM malic acid and an arginine acid aqueous solution at a ratio of 0.5 wt %. This running buffer was passed through the capillary channel at the same pressure as described above, and thereby the B layer was formed on the A layer. With the capillary channel being filled with the running buffer, a sample containing hemoglobin dissolved in distilled water was injected into the capillary channel. Thereafter, a voltage of 10 kV was applied across both ends of the capillary channel, and thereby electrophoresis was carried out. The hemoglobin-containing sample was injected into the capillary channel from the anode side thereof. The hemoglobin that had been transferred was detected at an absorbance of 415 nm. This result is shown in the electropherogram in FIG. 10. As shown in FIG. 10, in this example, it was possible to detect normal hemoglobin (HbA0) and glycated hemoglobin (HbA1c) separately. Furthermore, as for the capillary channel used in this example, because the B layer was formed simply by passing through the running buffer therein after being washed, it was possible to carry out the analysis immediately.

### Example 11

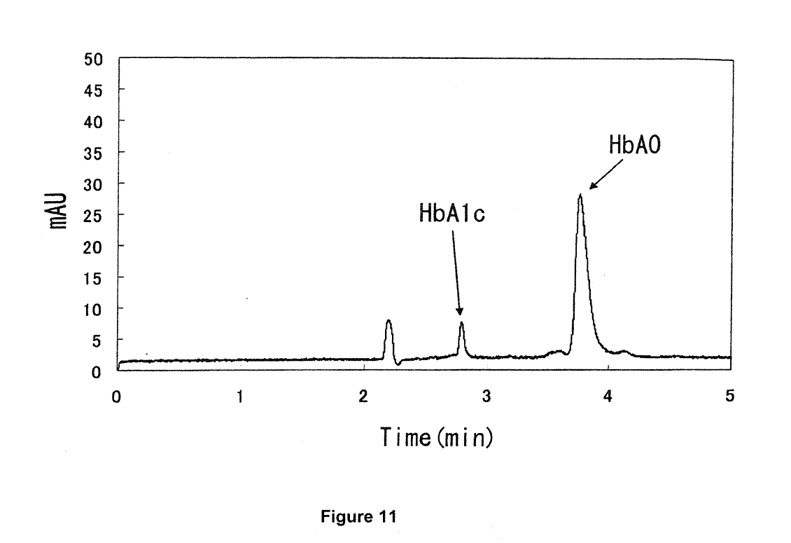

A capillary channel (with an overall length of 32 cm, an effective length of 8.5 cm, and an inner diameter of 50 μm) made of fused silica was prepared. The capillary channel had an A layer formed with poly(dimethylsiloxane) that was fixed to the inner wall thereof by a covalent bond. Distilled water was passed through this capillary channel at a pressure of 0.1 MPa (1000 mbar) for 20 minutes to wash it. Then, a running buffer (pH 5.5) was prepared that contains chondroitin sulfate added to 100 mM malic acid and an arginine acid aqueous solution at a ratio of 1.0 wt %. This running buffer was passed through the capillary channel at the same pressure as described above, and thereby the B layer was formed on the A layer. With the capillary channel being filled with the running buffer, a sample containing hemoglobin dissolved in distilled water was injected into the capillary channel. Thereafter, a voltage of 10 kV was applied across both ends of the capillary channel, and thereby electrophoresis was carried out. The hemoglobin-containing sample was injected into the capillary channel from the anode side thereof. The hemoglobin that had been transferred was detected at an absorbance of 415 nm. This result is shown in the electropherogram in FIG. 11. As shown in FIG. 11, in this example, it was possible to detect normal hemoglobin (HbA0) and glycated hemoglobin (HbA1c) separately. Furthermore, as for the capillary channel used in this example, because the B layer was formed simply by passing through the running buffer therein after being washed, it was possible to carry out the analysis immediately. In this state, the same analysis was carried out 10 times with the same sample as described above to evaluate repeatability. This result is shown in the following Table 2. In Table 2, as same as in Table 1, a relative area (%) denotes a ratio (%) of each peak area of the normal hemoglobin (HbA0) and the glycated hemoglobin (HbA1c) relative to a total peak area. As shown in Table 2, a value of coefficient of variation (CV) is small in each of the normal hemoglobin (HbA0) and the glycated hemoglobin (HbA1c). Thereby, it can be said that the analytical processes of the present invention is excellent in the precision.

TABLE 2

Relative Area (%)

| No. | HbA1c | HbA0 |
|---|---|---|
| 1 | 10.06 | 89.94 |
| 2 | 10.87 | 89.13 |
| 3 | 9.68 | 90.32 |
| 4 | 10.08 | 89.92 |
| 5 | 9.45 | 90.55 |
| 6 | 10.48 | 89.52 |
| 7 | 10.87 | 89.13 |
| 8 | 10.88 | 89.12 |
| 9 | 9.20 | 90.80 |
| 10 | 10.17 | 89.83 |
| Average | 10.17 | 89.83 |
| Coefficient of | 5.9 | 0.7 |
| Variation (CV) | | |

### Example 12

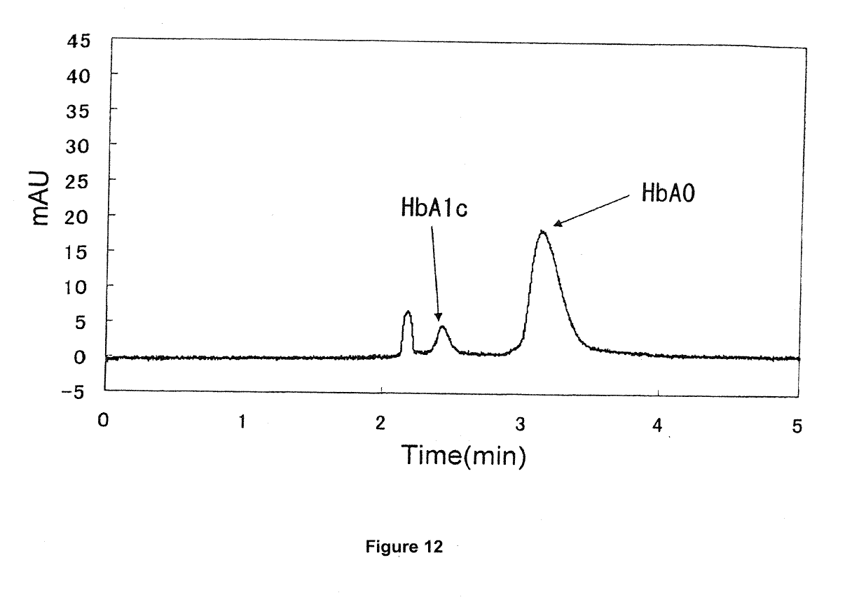

A capillary channel (with an overall length of 32 cm, an effective length of 8.5 cm, and an inner diameter of 50 μm) made of molten silica was prepared. The capillary channel had a cathode layer formed with a silylation agent having a sulfone group that was fixed to the inner wall thereof by a covalent bond. Purified water was passed through this capillary channel at a pressure of 0.1 MPa (1000 mbar) for 20 minutes to wash it. Subsequently, a running buffer (pH 5.5) was prepared that contains chondroitin sulfate added to 100 mM malic acid and an arginine acid aqueous solution at a ratio of 0.5 wt %. This running buffer was passed through the capillary channel at the same pressure as described above. With the capillary channel being filled with the running buffer, a sample containing hemoglobin dissolved in purified water was injected into the capillary channel. Thereafter, a voltage of 10 kV was applied to both ends of the capillary channel, and thereby electrophoresis was carried out. The hemoglobin-containing sample was injected into the capillary channel from the anode side thereof. The hemoglobin that had been transferred was detected at an absorbance of 415 nm. This result is shown in the chart in FIG. 12. As shown in FIG. 12, in this example, it was possible to detect normal hemoglobin (HbA0) and glycated hemoglobin (HbA1c) separately. Furthermore, the capillary channel used in this example allowed the analysis to be carried out immediately after being washed.

### Example 13

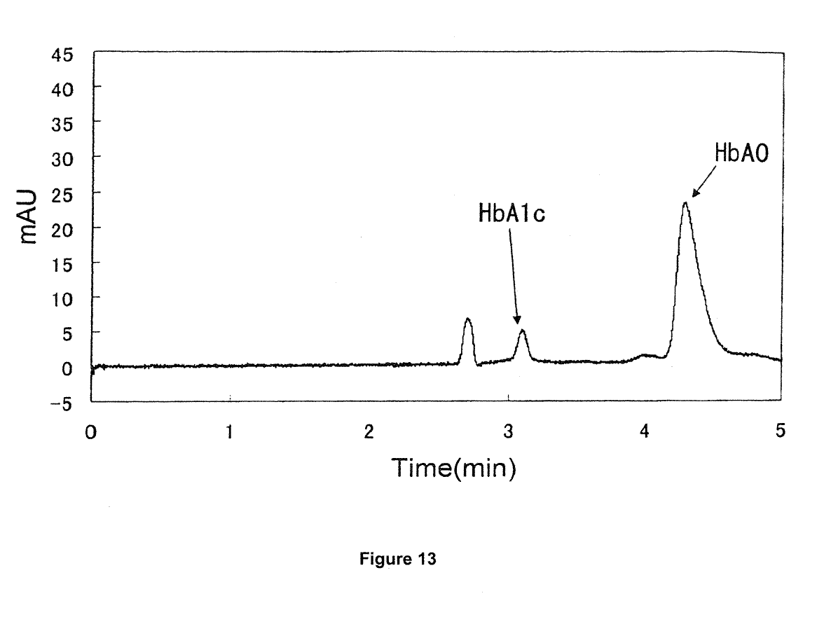

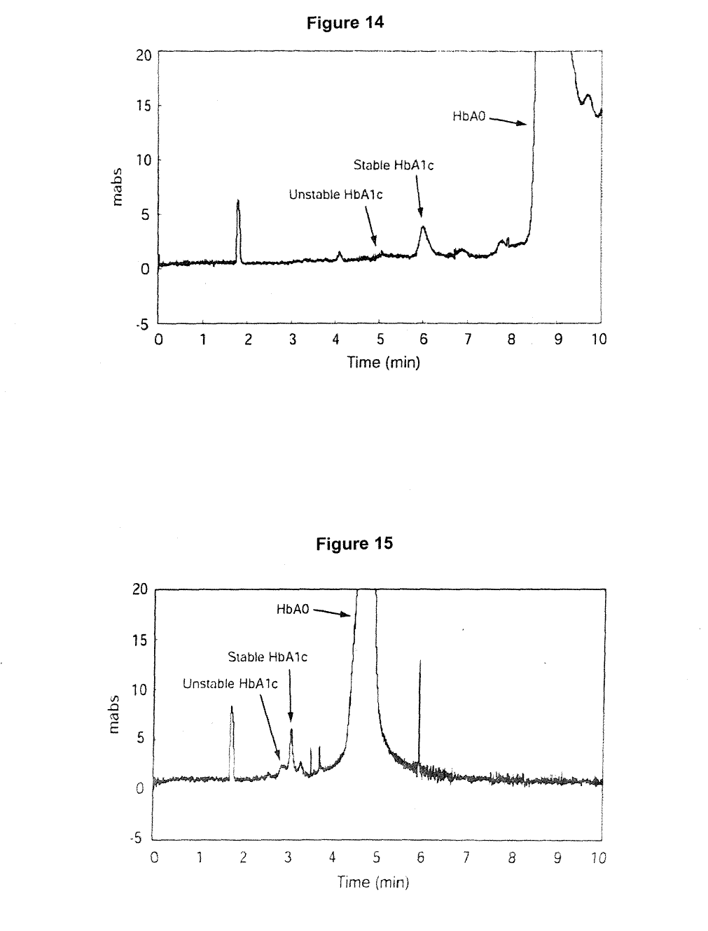

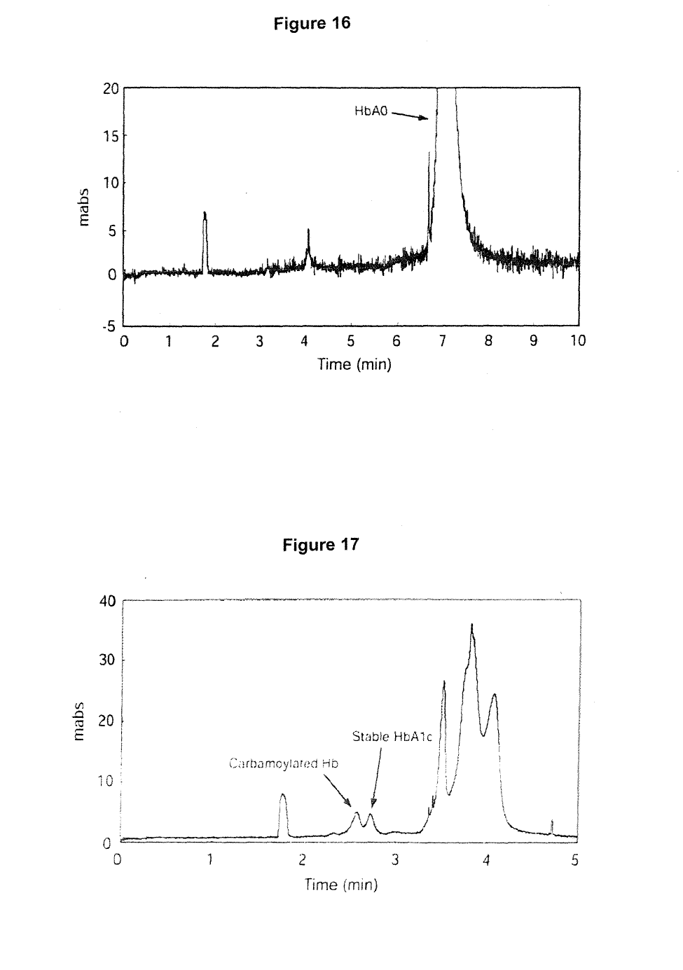

Hemoglobin was analyzed by CE carried out in the same manner as in Example 12 except that a capillary channel (with an overall length of 32 cm, an effective length of 8.5 cm, and an inner diameter of 50 μm) made of molten silica that had a cathode layer formed with a silylation agent having a carboxyl group that was fixed to the inner wall thereof by a covalent bond. This result is shown in the chart in FIG. 13. As shown in FIG. 13, in this example, it was possible to detect normal hemoglobin (HbA0) and glycated hemoglobin (HbA1c) separately. Furthermore, the capillary channel used in this example allowed the analysis to be carried out immediately after being washed.

### Example 14

### Comparative

The analysis was performed as in Example 1 except that perchloric acid was not added to the buffer solution.

### Example 15

### Comparative

The analysis was performed as in Example 1 except that guanidine (a cationic chaotropic ion), instead of perchloric acid, was added to the buffer solution to a concentration of 30 mmol/L.

### Example 16

### Comparative

The analysis was performed as in Example 1 except that urea (a neutral chaotropic ion), instead of perchloric acid, was added to the buffer solution to a concentration of 30 mmol/L.

### Example 17

The analysis was performed as in Example 1 except that a hemoglobin-containing sample was prepared by adding sodium cyanate at a concentration of 30 mg/100 mL to whole human blood.

### Example 18

### Comparative

The analysis method was performed as in Example 17 except that the perchloric acid was not added to the buffer solution.

### Example 19

The analysis was performed as in Example 1 except that acetaldehyde was added to whole human blood at a concentration of 30 mg/100 mL to prepare a hemoglobin-containing sample, instead of glucose.

### Example 20

### Comparative

The analysis was performed as in Example 19 except that perchloric acid was not added to the buffer solution.

With respect to Examples 1 to 6 in which a chaotropic anion was added to the buffer solution, each peak for stable HbA1c was detected as separated from the unstable HbA1c and HbA0 peaks. Further, the peaks for stable HbA1c, unstable HbA1c, and HbA0 were all detected within 5 minutes of beginning electrophoresis. In contrast, in Example 14 (Comparative) in which a chaotropic anion was not added to the buffer solution, the peak width of unstable HbA1c was increased, and the peak for unstable HbA1c could not be separated (e.g., resolved) from the peak for stable HbA1c. Further, the peak appeared slowly, and about 10 minutes were required before the HbA0-peak was detected in Example 14. With respect to Example 15 (Comparative) in which guanidine, which is a cationic chaotropic ion, was added to the buffer solution, the peak width for unstable HbA1c was increased, and the peak for unstable HbA1c could not be separated from the peak for stable HbA1c. With respect to Example 16 (Comparative) in which urea, which is a neutral chaotropic ion, was added to the buffer solution, both stable HbA1c and unstable HbA1c peaks could not be separated from the peak for HbA0. As described above, addition of chaotropic anion improves separation of stable HbA1c from unstable HbA1c and HbA0 and to significantly reduce the measurement time.

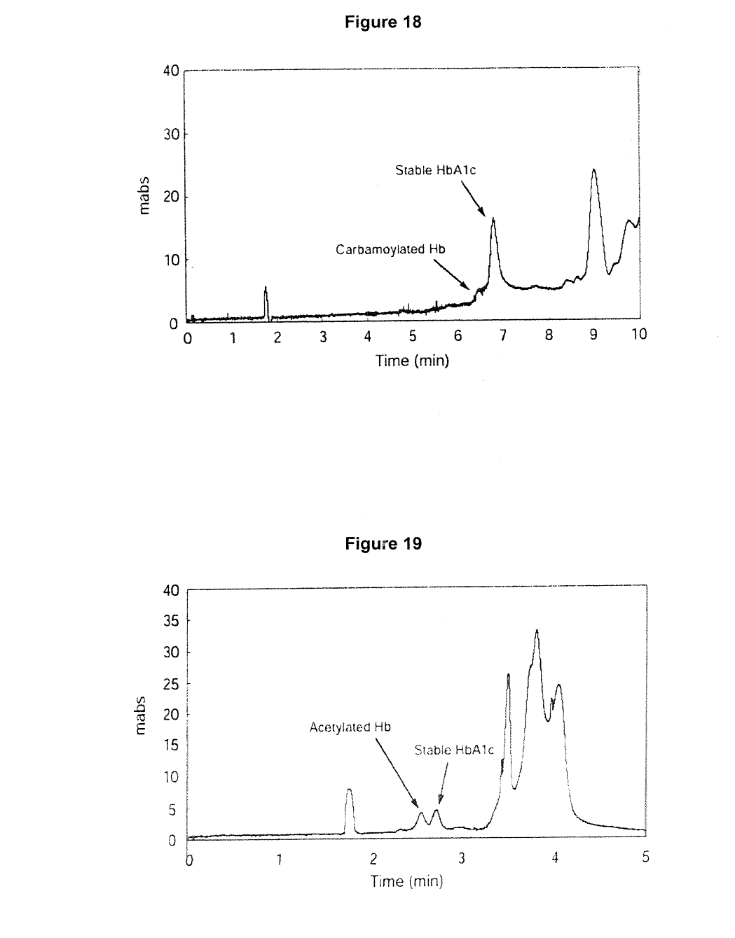

The results of Example 2 are shown in FIG. 17 and the results of Comparative Example 2 are shown in FIG. 18. In each graph of FIGS. 17 and 18, the vertical (y-) axis corresponds to absorbance measured at 415 nm and the horizontal axis corresponds to time in minutes. Further, in each of FIGS. 17 and 18, the peaks indicated by arrows, from left to right, are for carbamoylated Hb and stable HbA1c, respectively.

As shown in FIG. 17, in Example 2, the peak for stable HbA1c was separated from the peak for carbamoylated Hb. Further, in Example 2, the peaks for stable HbA1c and carbamoylated Hb were detected within 3 minutes from the start of electrophoresis and separation and detection could be performed relatively quickly. In contrast, as shown in FIG. 18, in Comparative Example 2, the peak for carbamoylated Hb could not be separated from the peak for stable HbA1c. Further, in Comparative Example 2, 7 minutes were required for the detection of the peak for stable HbA1c. As described above, addition of the chaotropic anion improves separation of stable HbA1c from carbamoylated Hb and to significantly reduce the time required to perform the analysis.

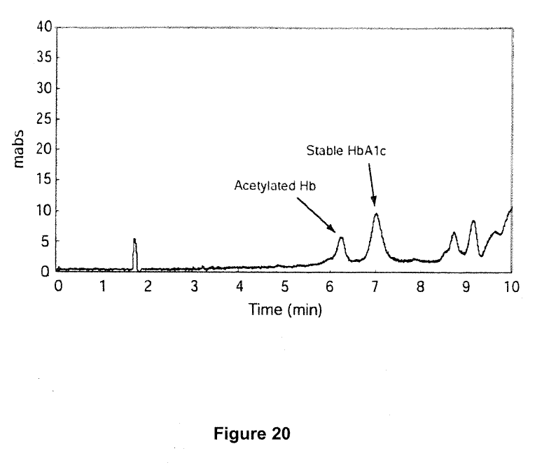

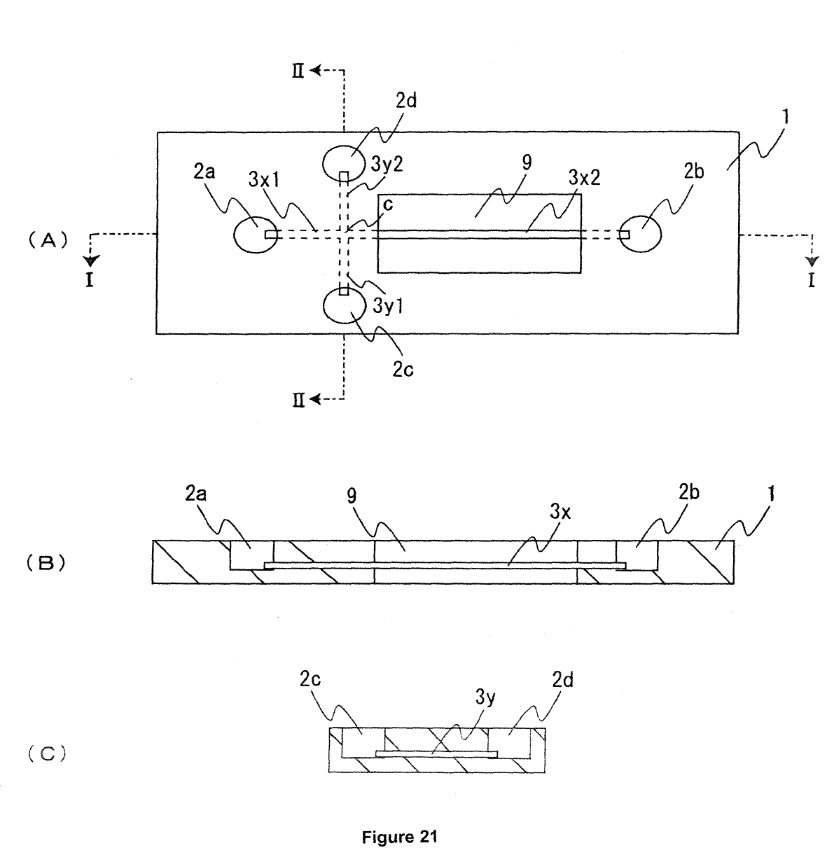

The results of Example 3 are shown in FIG. 19 and the results of Comparative Example 3 are shown in FIG. 20. In each graph of FIGS. 19 and 20, the vertical (y-) axis corresponds to the absorbance measured at 415 nm, and the horizontal (x-) axis corresponds to time in minutes. Further, in each of FIGS. 19 and 20, the peaks indicated by arrows, from left to right, are for acetylated Hb and stable HbA1c, respectively.

As shown in FIGS. 19 and 20, in Example 3 and Comparative Example 3, each peak for stable HbA1c was separated from peaks for acetylated Hb. Further, as shown in FIG. 19, in Example 3, the two peaks were detected within 3 minutes from the start of electrophoresis. In contrast, as shown in FIG. 20, in Comparative Example 3, the peaks appeared slowly and each peak for acetylated Hb and stable HbA1c was detected more than 6 minutes after electrophoresis was begun. As described above, addition of the chaotropic anion significantly reduces the time required to perform the analysis.

Methods for analyzing hemoglobin of the present invention, yield results with high accuracy, reduce analysis times, and the instrumentation requires less lab space than conventional methods. Certain aspects of the present invention may be used in clinical applications, biochemical studies, and medical research, among others.

It should be understood that the foregoing discussion and examples merely present a detailed description of certain preferred embodiments. It therefore should be apparent to those of ordinary skill in the art that various modifications and equivalents can be made without departing from the spirit and scope of the invention. All journal articles, other references, patents, and patent applications that are identified in this patent application are incorporated by reference, each in their entirety.

1. A capillary channel, wherein the inner wall of the capillary channel is coated with a coating comprising a cationic layer or an anionic layer.

2. The capillary channel of claim 1, wherein the cationic layer comprises amino groups or salts thereof, ammonium groups or mixtures thereof.

3. The capillary channel of claim 1, wherein the anionic layer comprises sulfate groups, carboxylate groups, sulfonate groups, phosphate groups or mixtures thereof.

4. The capillary channel of claim 1, wherein the anionic layer comprises a chaotropic anion.

5. The capillary channel of claim 1, wherein the inner diameter of the capillary channel is about 10 μm and about 200 μm.

6. A capillary channel, wherein the inner wall of the capillary channel is coated with a coating comprising an A layer and a B layer,
wherein the A layer comprises a cationic layer or a nonpolar layer, and the B layer comprises an anionic layer, and
wherein the B layer covers the A layer, the A layer being closer to the inner wall of the capillary channel than the B layer.

7. The capillary channel of claim 6, wherein the cationic layer comprises amino groups or salts thereof, ammonium groups or mixtures thereof.

8. The capillary channel of claim 6, wherein the cationic layer comprises a polydiallydimethylammonium group.

9. The capillary channel of claim 6, wherein the anionic layer comprises sulfate groups, carboxylate groups, sulfonate groups, phosphate groups or mixtures thereof.

10. The capillary channel of claim 6, wherein the anionic layer comprises an anionic group-containing polysaccharide.

11. The capillary channel of claim 10, wherein the polysaccharide of the anionic group-containing polysaccharide is a sulfated polysaccharide, a carboxylated polysaccharide, a sulfonated polysaccharide, a phosphorylated polysaccharide or mixtures thereof.

12. The capillary channel of claim 6, wherein the anionic layer comprises a chaotropic anion.

13. The capillary channel of claim 6, wherein the nonpolar layer comprises polysiloxanes, polysilazanes or mixtures thereof.

14. The capillary channel of claim 6, wherein the inner diameter of the capillary channel is about 10 μm and about 200 μm.

15. A capillary electrophoresis apparatus comprising the capillary channel of claim 1 or claim 6.

16. The capillary electrophoresis apparatus of claim 15, further comprising a substrate and a plurality of liquid tanks, wherein the liquid tanks are allowed to communicate with each other through the capillary channel.

17. A method of analyzing a sample comprising
applying a sample to the capillary electrophoresis apparatus of claim 15; and
performing electrophoretic separation of the sample, wherein the capillary channel contains an electrophoresis buffer solution.

18. The method of claim 17, wherein an anionic group-containing compound is present in the buffer solution, the sample or combinations thereof during at least a portion of the electrophoretic separation.

19. The method of claim 18, wherein the anionic group-containing compound is a chaotropic anion, a sulfated polysaccharide, a carboxylated polysaccharide, a sulfonated polysaccharide, a phosphorylated polysaccharide or mixtures thereof.

20. The method of claim 19, wherein the chaotropic anion is perchlorate, thiocyanate, trichloroacetate, trifluoroacetate, nitrate, dichloroacetate, halogenide or mixtures thereof.

21. The method of claim 17, wherein a chaotropic anion is present in the buffer solution, the sample or combinations thereof during at least a portion of the electrophoretic separation.

22. The method of claim 17, wherein the sample comprises hemoglobin.

23. A method of analyzing a sample comprising
applying a sample to a capillary electrophoresis apparatus comprising an uncoated capillary channel; and
performing electrophoretic separation of the sample, wherein the capillary channel contains an electrophoresis buffer solution and wherein an anionic group-containing compound is present in the buffer solution, the sample or combinations thereof during at least a portion of the electrophoretic separation.

24. The method of claim 23, wherein the anionic group-containing compound is a chaotropic anion.

25. The method of claim 23, wherein the anionic group-containing compound is a sulfated polysaccharide, a carboxylated polysaccharide, a sulfonated polysaccharide, a phosphorylated polysaccharide or mixtures thereof.

26. A method of diagnosing diabetes in a subject comprising
obtaining a sample of blood from a subject;
applying the sample to the capillary electrophoresis apparatus of claim 15; and
performing electrophoretic separation of the sample for determining the amount of glycated hemoglobin in the sample, thereby determining whether the subject has diabetes, wherein the capillary channel contains an electrophoresis buffer solution.

27. The method of claim 26, wherein an anionic group-containing compound is present in the buffer solution, the sample or combinations thereof during at least a portion of the electrophoretic separation.

28. The method of claim 27, wherein the anionic group-containing compound is a chaotropic anion, a sulfated polysaccharide, a carboxylated polysaccharide, a sulfonated polysaccharide, a phosphorylated polysaccharide or mixtures thereof.

29. The method of claim 28, wherein the chaotropic anion is perchlorate, thiocyanate, trichloroacetate, trifluoroacetate, nitrate, dichloroacetate, halogenide or mixtures thereof.

30. The method of claim 26, wherein a chaotropic anion is present in the buffer solution, the sample or combinations thereof during at least a portion of the electrophoretic separation.

31. A method of diagnosing diabetes in a subject comprising
obtaining a sample of blood from a subject;
applying the sample to a capillary electrophoresis apparatus comprising an uncoated capillary channel; and
performing electrophoretic separation of the sample for determining the amount of glycated hemoglobin in the sample, thereby determining whether the subject has diabetes, wherein the capillary channel contains an electrophoresis buffer solution and wherein an anionic group-containing compound is present in the buffer solution, the sample or combinations thereof during at least a portion of the electrophoretic separation.

32. The method of claim 31, wherein the anionic group-containing compound is a chaotropic anion.

33. The method of claim 31, wherein the anionic group-containing compound is a sulfated polysaccharide, a carboxylated polysaccharide, a sulfonated polysaccharide, a phosphorylated polysaccharide or mixtures thereof.

34. The method of claim 32, wherein the chaotropic anion is perchlorate, thiocyanate, trichloroacetate, trifluoroacetate, nitrate, dichloroacetate, halogenide or mixtures thereof.

35. A method of monitoring diabetes in a subject comprising
obtaining a sample of blood from a subject;
applying the sample to the capillary electrophoresis apparatus of claim 15; and
performing electrophoretic separation of the sample for determining the amount of glycated hemoglobin in the sample, thereby determining whether the subject has diabetes, wherein the capillary channel contains an electrophoresis buffer solution.

36. The method of claim 35, wherein an anionic group-containing compound is present in the buffer solution, the sample or combinations thereof during at least a portion of the electrophoretic separation.

37. The method of claim 36, wherein the anionic group-containing compound is a chaotropic anion, a sulfated polysaccharide, a carboxylated polysaccharide, a sulfonated polysaccharide, a phosphorylated polysaccharide or mixtures thereof.

38. The method of claim 37, wherein the chaotropic anion is perchlorate, thiocyanate, trichloroacetate, trifluoroacetate, nitrate, dichloroacetate, halogenide or mixtures thereof.

39. The method of claim 35, wherein a chaotropic anion is present in the buffer solution, the sample or combinations thereof during at least a portion of the electrophoretic separation.

40. A method of monitoring diabetes in a subject comprising
obtaining a sample of blood from a subject;
applying the sample to a capillary electrophoresis apparatus comprising an uncoated capillary channel; and
performing electrophoretic separation of the sample for determining the amount of glycated hemoglobin in the sample, thereby determining whether the subject has diabetes, wherein the capillary channel contains an electrophoresis buffer solution and wherein an anionic group-containing compound is present in the buffer solution, the sample or combinations thereof during at least a portion of the electrophoretic separation.

41. The method of claim 40, wherein the anionic group-containing compound is a chaotropic anion.

42. The method of claim 40, wherein the anionic group-containing compound is a sulfated polysaccharide, a carboxylated polysaccharide, a sulfonated polysaccharide, a phosphorylated polysaccharide or mixtures thereof.

43. The method of claim 41, wherein the chaotropic anion is perchlorate, thiocyanate, trichloroacetate, trifluoroacetate, nitrate, dichloroacetate, halogenide or mixtures thereof.

44. A kit for diagnosing or monitoring diabetes in a subject comprising a container for obtaining blood from a subject and at least one capillary electrophoresis buffer solution, wherein the buffer solution comprises an anionic group-containing compound.

45. The method of claim 44, wherein the anionic group-containing compound is a chaotropic anion.

46. The method of claim 44, wherein the anionic group-containing compound is a sulfated polysaccharide, a carboxylated polysaccharide, a sulfonated polysaccharide, a phosphorylated polysaccharide or mixtures thereof.

47. The method of claim 45, wherein the chaotropic anion is perchlorate, thiocyanate, trichloroacetate, trifluoroacetate, nitrate, dichloroacetate, halogenide or mixtures thereof.
